(19) United States
(12) Patent Application Publication
(10) Pub. No.: US 2024/0197623 A1
(43) Pub. Date: Jun. 20, 2024

(54) SILICA-BASED FORMULATIONS OF THERAPEUTIC OLIOGPEPTIDES AND PEPTIDOMIMETICS

(71) Applicant: Stealth Bio Therapeutics Inc., Needham, MA (US)

(72) Inventors: Martin P. Redmon, Boston, MA (US); Ari-Pekka Forsback, Turku (FI); Jari Mikkola, Kuusjoki (FI); Joona Sundqvist, Turku (FI)

(21) Appl. No.: 18/285,570

(22) PCT Filed: Apr. 7, 2022
(86) PCT No.: PCT/US2022/023828

§ 371 Date: Oct. 4, 2023

Related U.S. Application Data
(60) Provisional application No. 63/171,723, filed on Apr. 7, 2021.

(30) Foreign Application Priority Data
May 6, 2021 (FI) 20215537

(51) Int. Cl. A61K 9/00; A61K 9/06; A61K 9/16; A61K 31/4245; A61K 38/07; A61K 41/17; A61K 47/02; A61M 5/32

(52) U.S. Cl. CPC A61K 9/0024; A61K 9/06; A61K 9/1611; A61K 31/4245; A61K 38/07; A61K 41/17; A61K 47/02; A61M 5/32

(57) ABSTRACT

Disclosed are depot formulations comprising a biodegradable silica hydrogel composite, wherein the hydrogel composite comprises (i) a silica hydrogel; and (ii) a plurality of microparticles comprising silica and an oligopeptide or a peptidomimetic compound, or a pharmaceutically acceptable salt thereof. The depot formulations described herein provide sustained, long-term release of the active pharmaceutical compound. The depot formulations may be implanted in the eye and are useful for treating ocular diseases. The plurality of microparticles comprising silica and an oligopeptide or a peptidomimetic compound, or a pharmaceutically acceptable salt thereof can be prepared using a continuous flow process.

### RELATED APPLICATIONS

This application claims the benefit of priority to U.S. Provisional Patent Application No. 63/171,723, filed Apr. 7, 2021; and Finland Patent Application No. 20215537, filed May 6, 2021; both of which are incorporated herein by reference.

### BACKGROUND

Oligopeptides and peptidomimetic compounds are useful in treating a variety of diseases. For example, oligopeptides and peptidomimetic compounds have been used to treat diabetes, skin aliments, cardiac diseases, and obesity. Oligopeptides and peptiomimetic compounds also have demonstrated utility in treating a variety of ocular indications, such as Leber's Hereditary Optic Neuropathy and Fuchs' Corneal Endothelial Dystrophy. However, methods of administering these compounds to the eye remains an unmet challenge. Therefore, the need exists to develop formulations for and methods of administering oligopeptide and peptidomimetic compounds for ocular indications.

### SUMMARY

The present application provides depot formulations comprising a biodegradable silica hydrogel composite, wherein the hydrogel composite comprises a silica hydrogel and a plurality of microparticles comprising silica and an oligopeptide or a peptidomimetic compound, or a pharmaceutically acceptable salt thereof; wherein a freebase equivalent (defined below) of the oligopeptide or the peptidomimetic compound is less than 1,200 amu; and the hydrogel composite is non-flowing and structurally stable when stored at rest and shear-thinning when shear stress is applied. The depot formulation can be a controlled release depot, such as a zero-order release depot, first-order release depot, second-order release depot, delayed release depot, sustained release depot, immediate release depot, or any combination thereof.

In certain embodiments, the plurality of microparticles comprising silica and an oligopeptide or a peptidomimetic compound, or a pharmaceutically acceptable salt thereof, is formed using a continuous flow process.

In certain embodiments, the formulation is prepackaged in a syringe and needle configuration for single-use intravitreal injection.

In certain embodiments, the formulation is sterilized, e.g., by gamma ray radiation.

In another aspect, this application provides methods of treating, inhibiting, ameliorating or delaying the onset of a disease, disorder or condition, comprising administering to a subject in need thereof an effective amount of the depot formulation disclosed herein.

In certain embodiments, the condition is Leber's Hereditary Optic Neuropathy. In another aspect, this application provides methods of treating Fuchs endothelial corneal dystrophy (FECD), age-related macular degeneration (AMD), glaucoma, optic neuropathy, retinopathy, diabetic macular edema, diabetic retinopathy, retinal telangiectasia, or retinitis pigmentosa, comprising administering to a subject in need thereof an effective amount of the depot formulation disclosed herein. The age-related macular degeneration (AMD) can be wet AMD or dry AMD, or a combination thereof.

In another aspect, this application provides a prefilled syringe comprising a depot formulation disclosed herein. Optionally, the prefilled syringe is sterilized by exposure to gamma radiation.

The present application also provides methods of making the depot formulation disclosed herein, comprising combining:
a) silica particles comprising the oligopeptide or the peptidomimetic compound, or a pharmaceutically acceptable salt thereof, and having a maximum diameter of ≤1000 μm, optionally as a suspension, wherein a freebase equivalent of the oligopeptide or the peptidomimetic compound has a molecular weight less than 1,200 amu; and
b) a silica hydrogel;
   wherein:
i) the silica hydrogel has a solid content of ≤50 wt %;
ii) the silica particles comprise polymerized alkoxysilanes;
iii) the depot formulation comprises up to 85 wt % of said silica particles; and
iv) said hydrogel composite is non-flowing and structurally stable when stored at rest and shear-thinning when shear stress is applied by injection.

The methods of making the depot formulation may comprise one or more steps performed in a continuous flow process.

In certain embodiments, the methods further comprise sterilizing the depot formulation.

### BRIEF DESCRIPTION OF THE DRAWINGS

FIG. 1 is a graph showing the cumulative in-vitro dissolution under sink conditions of elamipretide depot formulation #05D.

FIG. 2 is a graph showing the cumulative in vitro in sink dissolution of elamipretide depot formulation #08D.

FIG. 3 is a graph showing the cumulative in vitro in sink dissolution of Compound I (defined below) depot formulation #05D.

FIG. 4 is a graph showing the cumulative in vitro in sink dissolution of Compound I (defined below) depot formulation #08D.

FIG. 5 is a flow diagram for production of depot formulations as packaged in pre-filled syringes as disclosed herein, wherein a continuous flow process is use to produce the plurality of microparticles comprising silica and an oligopeptide or a peptidomimetic compound, or a pharmaceutically acceptable salt thereof.

FIG. 6 is an illustration of the chemical polymerization of the hydrolyzed silica.

FIG. 7 shows a schematic of a continuous flow reactor system.

FIG. 8 shows a comparison of cumulative in vitro dissolution of the silica matrix (left) and the release of Compound I (right) from microparticle formulations #01-#03 with different 1^{st} R-values (2% API load, pH 5.9) in 50 mM TRIS buffer supplemented with 0.01% (v/v) TWEEN® 80 (pH 7.4 at 37° C. at in sink conditions. Mean values of triplicate analyses at each time point.

FIG. 9 shows a comparison of cumulative in vitro dissolution of the silica matrix (left) and the release of Compound I (right) from microparticle formulations #03-#06 with different pH (R3-100, 2% API load) in 50 mM TRIS buffer supplemented with 0.01% (v/v) TWEEN® 80 (pH 7.4 at 37° C. at in sink conditions. Mean values of triplicate analyses at each time point.

FIG. 10 shows a comparison of cumulative in vitro dissolution of the silica matrix (left) and the release of Compound I (right) from microparticle formulations #03, #05, #07 and #08 (R3-100 -based formulations) with different API load and pH in 50 mM TRIS supplemented with 0.01% (v/v) TWEEN® 80 (pH 7.4 at 37° C. at in sink conditions. Mean values of triplicate analyses at each time point.

FIG. 11 shows cumulative in vitro dissolution of the silica matrix and the release of Compound I from depot formulation #05D in 50 mM TRIS buffer supplemented with 0.01% (v/v) TWEEN® 80 (pH 7.4 at 37° C.). at in sink conditions. The dissolution profile for the corresponding microparticle formulation #05 (R3-100, 2% API load) is given as a comparison. Mean values of triplicate analyses at each time point.

FIG. 12 shows cumulative in vitro dissolution of the silica matrix and the release of Compound I from depot formulation #08D in 50 mM TRIS buffer supplemented with 0.01% (v/v) TWEEN® 80 (pH 7.4 at 37° C.) at in sink conditions. The dissolution profile for the corresponding microparticle formulation #08 (R3-100, 5% API load) is given as a comparison. Mean values of triplicate analyses at each time point.

FIG. 13 shows storage modulus (G′) and tan δ (loss factor) values for depot formulations #05D and #08D. The measurements were conducted under controlled deformation (γ<0.002) within the linear viscoelastic range of the materials. The reported values represent a single measurement.

FIG. 14 shows dynamic viscosity as a function of shear rate for depot formulations #05D and #08D. The reported values represent a single measurement.

FIG. 15 shows a comparison of cumulative in vitro dissolution of the silica matrix and the release of elamipretide from microparticle formulations #01-#03 with different 1^{st} R-values (2% API load, pH 5.9) in 50 mM TRIS buffer with 0.01% (v/v) TWEEN® 80 (pH 7.4 at 37° C. at in sink conditions. Mean values of triplicate analyses at each time point.

FIG. 16 shows a comparison of cumulative in vitro dissolution of the silica matrix and the release of elamipretide from microparticle formulations #03-#06 with different pH (R3-100, 2% API load) in 50 mM TRIS buffer supplemented with 0.01% (v/v) TWEEN® 80 (pH 7.4 at 37° C. at in sink conditions. Mean values of triplicate analyses at each time point.

FIG. 17 shows a comparison of cumulative in vitro dissolution of the silica matrix (left) and the release of elamipretide (right) from microparticle formulations #03, #05, #07 and #08 (R3-100 -based formulations) with different API load and pH in 50 mM TRIS buffer supplemented with 0.01% (v/v) TWEEN® 80 (pH 7.4 at 37° C.) at in sink conditions. Mean values of triplicate analyses at each time point.

FIG. 18 shows cumulative in vitro dissolution of the silica matrix and the release of elamipretide from depot formulation #05D in 50 mM TRIS buffer supplemented with 0.01% (v/v) TWEEN® 80 (pH 7.4 at 37° C.) at in sink conditions. The dissolution profile for the corresponding microparticle formulation #05 (R3-100, 2% API load) is given as a comparison. Mean values of triplicate analyses at each time point.

FIG. 19 shows cumulative in vitro dissolution of the silica matrix and the release of elamipretide from depot formulation #08D in 50 mM TRIS buffer with 0.01% (v/v) TWEEN® 80 (pH 7.4 at 37° C.) at in sink conditions. The dissolution profile for the corresponding microparticle formulation #08 (R3-100, 5% API load) is given as a comparison. Mean values of triplicate analyses at each time point.

FIG. 20 shows storage modulus (G′) and tan δ (loss factor) values for depot formulations #05D and #08D. The measurement was conducted under controlled deformation (γ<0.002) within the linear viscoelastic range of the materials. The reported values represent a single measurement.

FIG. 21 shows dynamic viscosity as a function of shear rate for depot formulations #05D and #08D. The reported values represent a single measurement.

FIG. 22 shows graphs of particle size distribution for Formulations #09 and #10-5 consistency batches.

FIG. 23 shows graphs of cumulative in vitro in sink dissolution of elamipretide microparticle formulation #09 and #10-5 consistency batches.

FIG. 24 shows graphs of particle size distribution for Formulations #09 and #10-5 continuous flow 10×batches.

FIG. 25 shows graphs of cumulative in vitro in sink dissolution of elamipretide microparticle formulation #09 and #10-5 continuous flow 10×batches.

### DETAILED DESCRIPTION

The present application relates to a depot formulation comprising a biodegradable silica hydrogel composite. The hydrogel composite comprises a silica hydrogel and a plurality of microparticles comprising silica and an oligopeptide or a peptidomimetic compound, or a pharmaceutically acceptable salt thereof, wherein the freebase equivalent of the oligopeptide or the peptidomimetic compound has a molecular weight less than 1,200 amu. The hydrogel composite is non-flowing and structurally stable when stored at rest, and shear-thinning when shear stress is applied, for example when force is applied to by injection via a syringe.

In some embodiments, the oligopeptide or the peptidomimetic compound of the depot formulation is positively charged in aqueous buffered solution at neutral pH (e.g., the net charge is greater than or equal to +2).

In some embodiments, the oligopeptide or the peptidomimetic compound of the depot formulation exhibits good water solubility (e.g., has a water solubility of >50 mg/mL, >75 mg/mL, >100 mg/mL or >150 mg/mL).

In some embodiments, the plurality of microparticles of the hydrogel composite is present in varying amounts. In some embodiments, the microparticles are present in the depot formulation in an amount from about 30 to about 95 wt %, or from about 45 to about 90 wt %, or from about 50 to about 85 wt %, or from 75 to 90 wt %. In some embodiments, the microparticles are present in the depot formulation in an amount of about 80 wt %. In some embodiments, the microparticles are present in the depot formulation in an amount from about 30 to about 70 wt %, or from about 40 to about 60 wt %, or from about 20 to about 40 wt %. In some embodiments, the microparticles are present in the depot formulation in an amount of about 50 wt %. In some embodiments, the microparticles are present in the depot formulation in an amount from about 20 to about 50 wt %, or 20 to about 40 wt %.

The depot formulation can comprise an oligopeptide, which can be present in a range of amounts. In some embodiments, the freebase equivalent of an oligopeptide in the depot formulation is present in an amount from about 0.1 wt % to about 10 wt %, or from about 0.3 wt % to about 5 wt %, or from about 1 wt % to about 3 wt % or from about 2 wt % to about 4 wt %. In some embodiments, the freebase equivalent of an oligopeptide in the depot formulation is present in an amount from about 2 wt % to about 5 wt %, or from about 0.25 wt % to about 2.5 wt %, or from 0.45 wt % to 2 wt %.

The depot formulation can comprise a peptidomimetic compound, which can be present in a range of amounts. In some embodiments, the freebase equivalent of a peptidomimetic compound in the depot formulation is present in an amount from about 0.1 wt % to about 10 wt %, or from about 0.3 wt % to about 5 wt %, or from about 1 wt % to about 3 wt % or from about 2 wt % to about 4 wt %. In some embodiments, the freebase equivalent of a peptidomimetic compound in the depot formulation is present in an amount from about 2 wt % to about 5 wt %, or from about 0.25 wt % to about 2.5 wt %, or from 0.45 wt % to 2 wt %.

In certain embodiments, the depot formulation is shear-thinning when sheer stress is applied, and may be injectable through thin needles (e.g. 18-30 gauge needles, 20-30 gauge needles, 25-30 gauge needles, or 27-30 gauge needles). In certain embodiments, when shear stress is applied, the silica hydrogel composite is free flowing and can be injected from a syringe having an attached needle. In certain embodiments, the needle thickness is 25-30 gauge. In some embodiments, the needle thickness is up to 25 gauge. In some embodiments, the needle thickness is up to 30 gauge. In some embodiments, the needle thickness is up to 35 gauge. In some embodiments, the needle thickness is up to 40 gauge. In some embodiments, the needle thickness is up to 50 gauge.

After injection (i.e., at the conclusion of the application of shear stress), the silica hydrogel composite is non-flowing and structurally stable at 37° C.

In some embodiments, the depot formulations disclosed herein can comprise a range of pH values. In some embodiments, the depot formulation has a pH of about 5.5 to about 7.5, or about 5.5 to about 7.0, or about 5.5 to about 6.5.

In some embodiments, the microparticles of the depot formulation can comprise more than one oligopeptide or peptidomimetic compound. In some embodiments, the oligopeptide or the peptidomimetic compound of the microparticles is mitochondria-targeted.

In some embodiments, the microparticles comprise an oligopeptide, or a pharmaceutically acceptable salt thereof. In some embodiments, the oligopeptide is selected from the group consisting of elamipretide, Compound II, Compound III, and Compound IV, and pharmaceutically acceptable salts thereof. The structures of elamipretide, Compound II, Compound III, and Compound IV (in freebase equivalent forms) are depicted below:

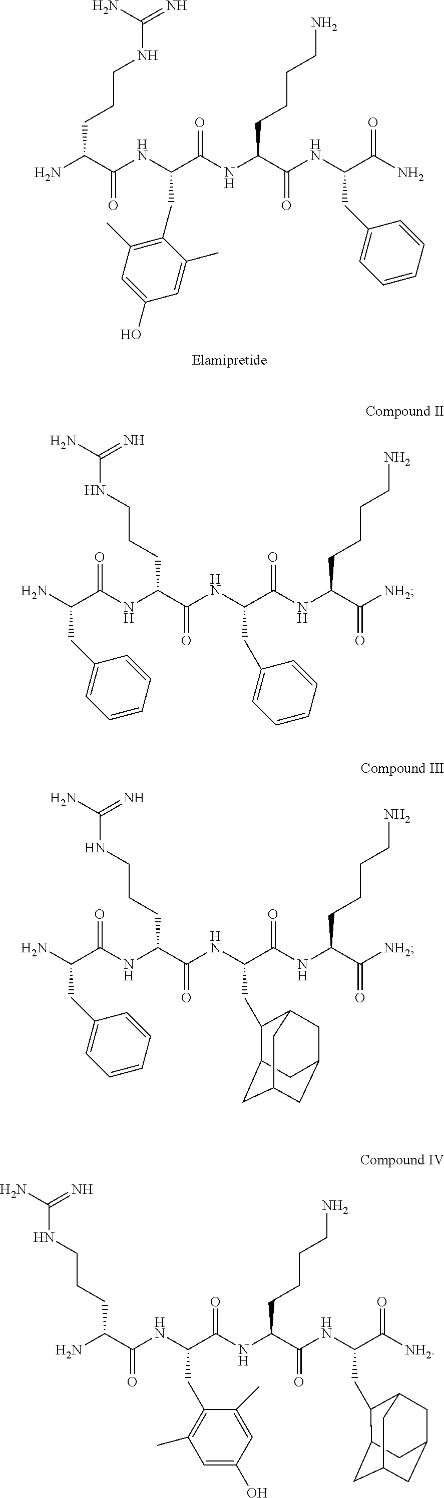 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O ; N=C(N)NCCC[C@@H](NC(=O)[C@@H](N)Cc1ccccc1)C(=O)N[C@@H](Cc1ccccc1)C(=O)N[C@@H](CCCCN)C(N)=O ; N=C(N)NCCC[C@@H](NC(=O)[C@@H](N)Cc1ccccc1)C(=O)N[C@@H](CC1C2CC3CC(C2)CC1C3)C(=O)N[C@@H](CCCCN)C(N)=O ; Cc1cc(O)cc(C)c1C[C@H](NC(=O)[C@H](N)CCCNC(=N)N)C(=O)N[C@@H](CCCCN)C(=O)N[C@@H](CC1C2CC3CC(C2)CC1C3)C(N)=O]

In certain embodiments, the oligopeptide is elamipretide, or a pharmaceutically acceptable salt thereof.

In certain embodiments, the oligopeptide is Compound II, or a pharmaceutically acceptable salt thereof.

In certain embodiments, the oligopeptide is Compound III, or a pharmaceutically acceptable salt thereof.

In certain embodiments, the oligopeptide is Compound IV, or a pharmaceutically acceptable salt thereof.

In some embodiments, the microparticles of the depot formulation can comprise a peptidomimetic compound, or a pharmaceutically acceptable salt thereof. Compound I is an example of one such peptidomimetic compound (or a pharmaceutically acceptable salt thereof) that can be used in depot formulations disclosed herein:

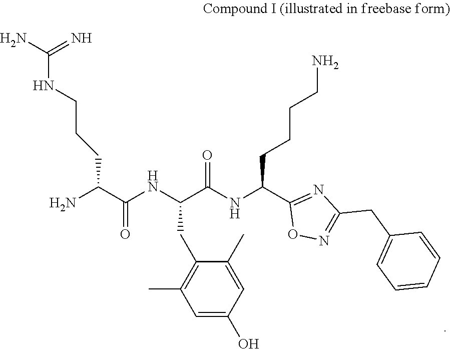 [SMILES: Cc1cc(O)cc(C)c1C[C@H](NC(=O)[C@H](N)CCCNC(=N)N)C(=O)N[C@@H](CCCCN)c1nc(Cc2ccccc2)no1]

The depot formulations disclosed herein can be formulated to be suitable for injection, implantation, inhalation, or other means of parenteral administration. In some aspects, the depot formulation can be formulated to be suitable for subcutaneous, intramuscular, peritoneal, or ocular administration. In some embodiments, the depot formulation is formulated for intravitreal injection or topical ocular delivery of the oligopeptide or the peptidomimetic compound. In certain embodiments, the formulation is for intravitreal injection. In some embodiments, the depot formulation is formulated for intravenous or subcutaneous administration for systemic delivery of the oligopeptide or the peptidomimetic compound.

In some embodiments, the depot formulation is for administration from once a week to once a year, or once a month to once a year, or once a month to once every six months, or once a week to once a month, or once a month to once every three months, once every three months to once every six months, once every six months to once every nine months, or once every nine months to once per year. Alternatively, the depot formulation may be for administration from once a month to once every six months, or once a month to once every five months, once every two months to once every five months, once every two months to once every four months, or once every two months to once every three months, once every three months to once every four months, or once about every three months.

Depot formulations that release drug over time are referred to as “controlled release” depot formulations. Such depot formulations formulated for controlled release may aid in patient compliance, since for some patients it is easier to have the injection once per an extended period of time than to take a medication daily. In further embodiments, a controlled release depot formulation may be better tolerated by a patient as compared to, e.g., daily injections (e.g. daily subcutaneous injections), since the depot formulation will be associated with less frequent injection site reactions.

In some embodiments, the formulation delivers a daily dose of the oligopeptide or the peptidomimetic compound of from 0.01 μg to 10 μg, from 0.1 μg to 5 μg, from 1 μg to 5 μg, or about 3 μg, wherein the amounts stated are calculated based on the freebase equivalent mass of the oligopeptide or peptidomimetic compound (regardless of whether a freebase or salt form of the oligopeptide or the peptidomimetic compound is used in preparation of the formulation).

In some embodiments, the amount of the freebase equivalent of the oligopeptide or the peptidomimetic compound in the depot formulation is from 10 μg/50 μL to 100 mg/50 μL, from 10 μg/50 μL to 1000 μg/50 μL, from 10 μg/50 μL to 500 μg/50 μL, from 50 μg/50 μL to 300 μg/50 μL, or from 75 μg/50 μL to 275 μg/50 μL, wherein the amounts stated are calculated based on the freebase equivalent mass of the oligopeptide or peptidomimetic compound. Alternatively, the amount of the freebase equivalent of the oligopeptide or the peptidomimetic compound in the depot formulation is from 100 μg/50 μL to 500 μg/50 μL, from 150 μg/50 μL to 450 μg/50 μL, from 200 μg/50 μL to 400 μg/50 μL, from 250 μg/50 μL to 350 μg/50 μL, or about 270 μg/50 μL.

In certain embodiments, the depot formulation is for intravitreal injection about every three months.

In certain embodiments, the depot formulation is sterilized, e.g., by gamma ray sterilization.

Depot formulations of the present application can comprise a silica hydrogel with a range of solid content. In some embodiments, the silica hydrogel has a solid content of ≤3 wt %, ≤2 wt % or ≤1 wt %. In some embodiments, the silica particles comprise from 0.1 to 70 wt %, from 0.3 to 50 wt %, or from 1 to 15 wt % of the oligopeptide or the peptidomimetic compound.

In some embodiments of the depot formulation the plurality of microparticles comprises silica particles with a range of diameters. In some embodiments, the silica particles have a diameter of between 0.5 μm and 300 μm. In some embodiments, the silica particles have a diameter of between 0.5 μm and 100 μm, between 0.5 μm and 30 μm or between 0.5 μm and 20 μm.

The depot formulation of the present application can have a volume fraction of silica particles in a range of diameters. In some embodiments the volume fraction of silica particles with a diameter <1 μm is <3%, <2%, or <1%.

In some embodiments, the depot formulation can comprises a composite solid content from 10 wt % to 75 wt %, from 15 wt % to 60 wt % or from 25 wt % to 55 wt %.

In some embodiments, the depot formulation comprises a range of complex modulus measured under small angle oscillatory shear in the linear viscoelastic region. In some embodiments, the complex modulus is <2400 kPa, <1200 kPa or <600 kPa.

In some embodiments, the depot formulation comprises a loss factor (i.e., viscous modulus/elastic modulus) value of <1, <0.8, or <0.6.

In some embodiments, the depot formulation has a viscosity of about 10-50 Pas measured with a shear rate of 10-50 s⁻¹, a viscosity of about 0.4-1.5 Pas measured with a shear rate of 200-210 s⁻¹, or a viscosity of about 0.1-0.4 Pas measured with a shear rate of 600-610 s⁻¹.

In certain embodiments, the formulation is prepackaged in a syringe and needle configuration for single-use intravitreal injection.

The present application also provides a prefilled syringe comprising the depot formulation described herein. In some embodiments, the prefilled syringe has been sterilized by exposure to gamma radiation.

Microparticles can be prepared by many different methods. Microparticles (often, but not necessarily, spherical particles, microspheres) are typically small particles with diameters in the micrometer range (typically 1 μm to 1000 μm). There are several direct techniques that are commonly used to prepare or manufacture microparticles with a controlled particle size distribution, such as spray-drying (often followed by centrifugal separation of the microparticles in cyclones), single emulsion, double emulsion, polymerization, coacervation phase separation and solvent extraction methods. By using these techniques, a small volume fraction of submicron particles (commonly between 0.5-1.0 μm) may be included in the resulting product, but their proportion is usually very low, often less than 1 volume-%. Thus, their effect, e.g., in controlled release microparticle formulations, is minor. It is also possible to prepare microparticles by casting or by crushing larger structures to microparticles, but in that case the size and form of the resulting particles may vary and additional preparation steps, such as tumbling and particle sizing may be needed.

In certain embodiments, the plurality of microparticles comprising silica and an oligopeptide or a peptidomimetic compound, or a pharmaceutically acceptable salt thereof, is formed using a continuous flow process.

In certain embodiments, the continuous flow process further comprises use of a spray dryer to generate the plurality of microparticles comprising silica and an oligopeptide or a peptidomimetic compound, or a pharmaceutically acceptable salt thereof. In further embodiments, the continuous flow process further comprises use of an in-line mixer.

Silica microparticles of the silica-silica composites have a role in the controlled release of drugs and the hydrogel structure ensures both stability and injectability of the resulting composite. Although stable at rest, e.g., as stored in a prefilled syringe, the hydrogel composite structure is so loose that it is shear-thinning when the hydrogel composite is injected from ready-to-use syringes through thin needles (e.g., 20-30 gauge, such as 20 gauge, 24 gauge, 25 gauge, 28 gauge or 30 gauge). This combination of properties provides a minimally invasive, long-term treatment of, e.g., an ocular disease.

The present application further provides a method of treating, inhibiting, ameliorating or delaying the onset of other diseases, disorders or conditions such as Fuchs endothelial corneal dystrophy (FECD), (wet or dry) age-related macular degeneration (AMD), glaucoma, optic neuropathy, retinopathy, diabetic macular edema, diabetic retinopathy, retinal telangiectasia, or retinitis pigmentosa. The method comprising administering to a subject in need thereof an effective amount of the depot formulation is described herein. In some embodiments, the AMD is neovascular AMD (wet AMD) or retinal geographic atrophy (dry AMD).

The present application further provides a method of treating, inhibiting, ameliorating or delaying the onset of a disease, comprising administering to a subject in need thereof an effective amount of the depot formulation described herein. The disease to be treated can be Leber's Hereditary Optic Neuropathy.

In some embodiments, the disease may be associated with mitochondrial dysfunction.

The present application further provides a method of making a depot formulation described herein, comprising the step of combining: silica particles comprising the oligopeptide or the peptidomimetic compound, or a pharmaceutically acceptable salt thereof, and having a maximum diameter of ≤1000 μm, optionally as a suspension, wherein a freebase equivalent of the oligopeptide or the peptidomimetic compound has a molecular weight less than 1,200 amu; with a silica hydrogel. In some embodiments, the silica hydrogel has a solid content of ≤50 wt %; the silica particles comprise polymerized alkoxysilanes; the depot formulation comprises up to 85 wt % of said silica particles; and said hydrogel composite is non-flowing and structurally stable when stored at rest and shear-thinning when shear stress is applied by injection.

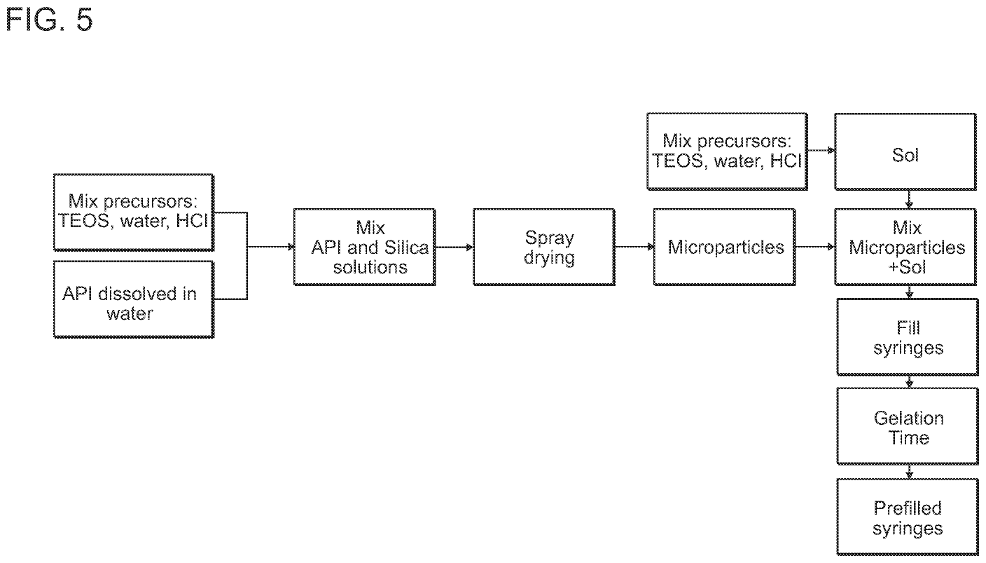

In some embodiments, the method further comprises combining a silica sol with a solution comprising the oligopeptide or the peptidomimetic compound, or a pharmaceutical salt thereof, thereby producing a mixture comprising the silica sol and the oligopeptide or a peptidomimetic compound, or a pharmaceutically acceptable salt thereof. The resulting mixture may be spray dried to form the silica particles that are ultimately combined with the silica hydrogel (see for example, FIG. 5).

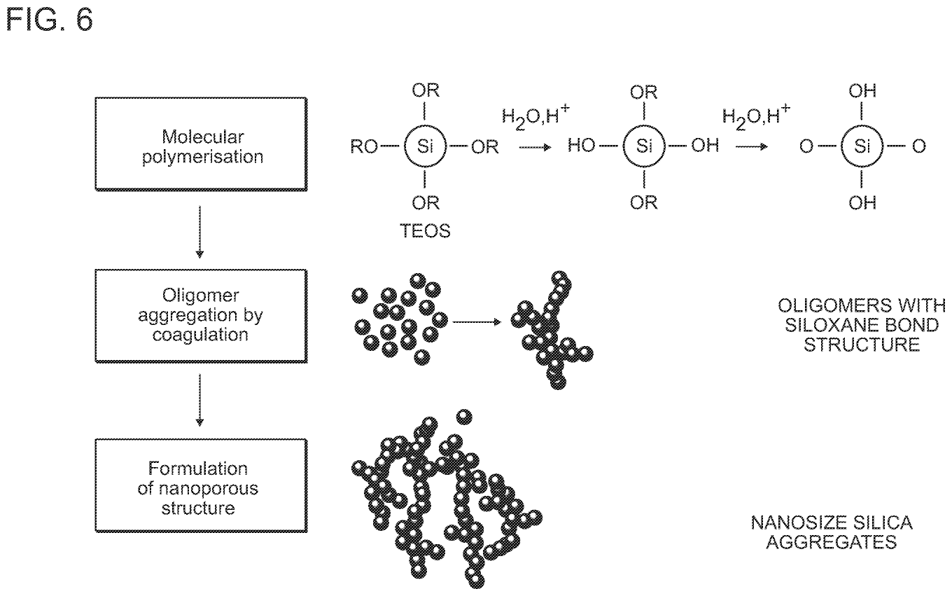

In certain such embodiments, the method further comprises combining tetraethyl orthosilicate (TEOS), water and hydrochloric acid, and optionally an organic co-solvent (e.g. ethanol), thereby producing the silica sol (see for example, FIGS. 5 and 6).

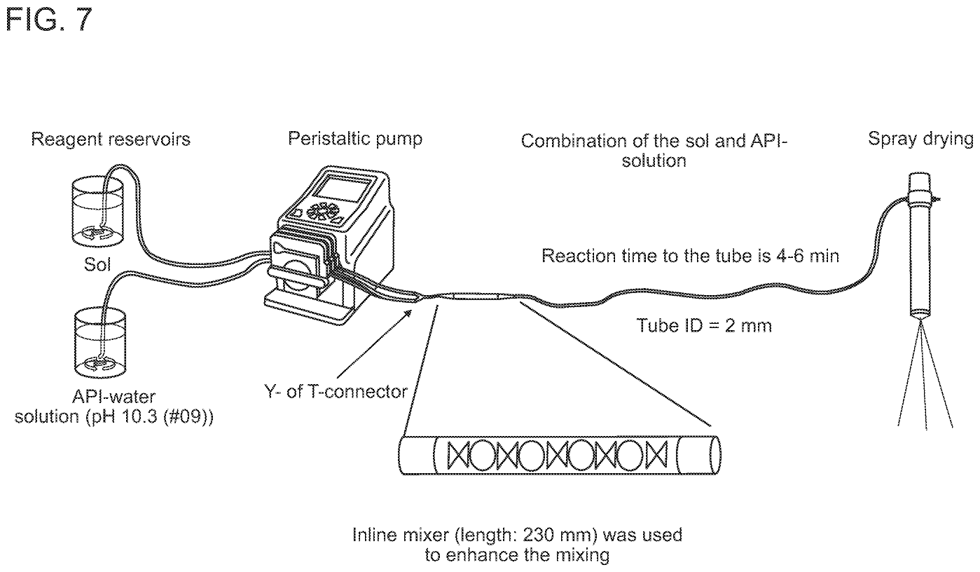

In further such embodiments, the method comprises combining the oligopeptide or the peptidomimetic compound, or a pharmaceutical salt thereof, with water and optionally an organic co-solvent (e.g. ethanol), thereby producing the solution comprising the oligopeptide or the peptidomimetic compound, or a pharmaceutical salt thereof (e.g. the API solution, See for example, FIG. 7).

In certain embodiments, the step of combining the silica sol and the solution comprising the oligopeptide or the peptidomimetic compound, or a pharmaceutical salt thereof, is performed in a continuous flow process, e.g., in a continuous flow reactor. Such a process may comprise use of an in-line mixer and/or a pump (see, for example, FIGS. 5-7).

The continuous flow process is repeatable and enables scale-up. Accordingly, in some embodiments of the method of making the depot formulation, the formulation is produced on 10×scale, 50×scale, 150×scale, 250×scale, 500×scale, 750×scale, 1000×scale, 1500×scale, 2000×scale, or higher. The continuous flow process can provide for more consistent particle size (and size distribution) and API content as compared to production methods using a single batch reactor.

In certain embodiments, the formulation is packaged into a syringe, optionally with an attached needle.

The method of making the depot formulation may further comprise sterilizing the depot formulation, e.g., by radiation sterilization. The radiation sterilization may comprise gamma-ray radiation sterilization.

In certain embodiments, the oligopeptide is elamipretide or a pharmaceutically acceptable salt thereof.

In certain embodiments, the oligopeptide is Compound II or a pharmaceutically acceptable salt thereof.

In certain embodiments, the oligopeptide is Compound III or a pharmaceutically acceptable salt thereof.

In certain embodiments, the oligopeptide is Compound IV or a pharmaceutically acceptable salt thereof.

In certain embodiments, the peptidomimetic is Compound I or a pharmaceutically acceptable salt thereof.

### Definitions

As used herein, to “ameliorate” or “ameliorating” a disease, disorder or condition (e.g., a tauopathy) refers to results that, in a statistical sample or specific subject, make the occurrence of the disease, disorder or condition (or a sign, symptom or condition thereof) better or more tolerable in a sample or subject administered a therapeutic agent relative to a control sample or subject.

The term biodegradation refers to erosion, i.e., to gradual degradation of the matrix material, e.g., silica in the body. The degradation occurs preferably by dissolution in the body fluids.

As used herein, the word “delays” or the phrase “delaying the onset of” refers to, in a statistical sample, postponing, hindering the occurrence of a disease, disorder or condition, or causing one or more signs, symptoms or conditions of a disease, disorder or condition to occur more slowly than normal, in a sample or subject administered a therapeutic agent relative to a control sample or subject.

Depot formulation referred to in the application is defined to be the administration of a controlled release drug (active agent) formulation that allows slow release and gradual absorption by the subject, so that the active agent can act and is released in the subject's body over extended periods of times, i.e., from several days to several months. Depot formulations are administered parenterally, either by subcutaneous, intramuscular, peritoneal or ocular implantation or injection.

Freebase equivalent should be understood to refer to a compound (e.g., an oligopeptide or peptidomimetic) in its simplest, lowest molecular weight form. For example, the form that does not include salts or other atoms, molecules or agents that might typically be associated with, or complexed to, the compound. For example, elamipretide and Compounds I-IV comprise three basic nitrogen atoms that typically will complex a negative ion when the nitrogen is protonated (and thereby rendered positive in charge). The freebase equivalents of elamipretide and Compounds I-IV are illustrated above.

Gel should be understood in the context of this application to be a homogeneous mixture of at least one solid phase and one liquid phase, i.e., a colloidal dispersion, where solid phase(s), e.g., silica as such and/or as partly or fully hydrolysed, is the continuous phase and the liquid(s), e.g., water, ethanol and residuals of silica precursors, is homogeneously dispersed in the structure. The gel is viscoelastic and the elastic properties dominate, which is indicated by rheological measurements under small angle oscillatory shear. The elastic properties dominate and the structure is non-flowing when the loss factor (or the loss tangent), tan δ=(G″/G′), is less than 1. The combined effect of the elastic modulus G′ and the viscous modulus G″ can also be expressed in the form of complex modulus (or complex shear modulus), G*=G′+iG″.

Gel point shall be understood to mean the time point when the sol that is flowing turns to a gel that is viscoelastic and the elastic properties dominate, which is indicated by rheological measurements under small angle oscillatory shear that the elastic modulus, G′is greater than the viscous modulus and the loss factor is less than 1. The viscoelastic properties are commonly measured with a rheometer (a measuring device for determination of the correlation between deformation, shear stress and time) by the oscillatory shear, where shear stresses are small (small angles of deformation). The total resistance in small oscillatory shear is described by the complex modulus (G*). The complex modulus (or complex shear modulus), contains two components (G*=G′+iG″): 1) elastic modulus, also called storage modulus, G′ that describes that material has some elastic properties that are characteristic for a solid material, i.e., the gel system will gain energy from the oscillatory motion as long as the motion does not disrupt the gel structure. This energy is stored in the sample and is described by elastic modulus; 2) viscous modulus, also called loss modulus, G″ that describes flow properties, i.e., a system, e.g., a silica sol that will in an oscillatory shear create motion between the ingredients of the sol describing the part of the energy, which is lost as viscous dissipation. As G*=G′ a material is called elastic and as G*=G″ a material is called viscous. At the gel point, the elastic modulus, G′ becomes larger than the viscous modulus, G″. As G′>G″, a viscoelastic material is called semisolid and correspondingly as G″>G, a viscoelastic material is called semi-liquid. The magnitude of the elastic and viscous modulus depends on the shear stress, which depends on the applied strain (small angle deformation) and frequency (of the oscillatory shear). The measurements are conducted by ensuring an adequate signal for a specific measuring system, i.e., a strain sweep is commonly done at constant frequencies to find the proper signal and the linear viscoelastic region for the rheometer system and then the actual measurements are done at constant strain with varying frequency. The varying frequencies give varying elastic and viscous modulus and the measurement show whether the solid or liquid phase dominates. It is also typical that the elastic modulus increases fast after the gel point if the surrounding conditions are not significantly changed, e.g., 100-700 fold increase in G′ within few minutes after the gel point is typical for gels formed from acidic sols near room temperature, e.g., for a R15 sol at pH=2 that turns to a gel (R=water-to-alkoxide molar ratio). For larger R-values, such as R150 and R400, the elastic modulus, G′ remains on a low level even after the gel point and increase of G′ is not fast, which makes it possible to have gel structures that remain injectable with thin needles. In the form of a gel after the defined gel point, the solid state dominates, but the system still contains varying amounts of liquids and the material is typically soft and viscoelastic before drying, and hard and brittle if it is extensively dried. In the form of a sol, the liquid state dominates, but the system contains varying amounts of solid phase(s) and the system is still flowing. Before the gel point it is typical that a steep increase in dynamic viscosity and elastic modulus is observed, which continues to rise after the gel point as the structure is developing. In the context of the present application gel point of the composite has been reached prior to obtaining the injectable gel.

The hydrogel should be understood to be a gel, where the liquid phase is water or water-based containing more than 50 weight-% (wt-%) of water. Typically the liquid phase of the hydrogel comprises >65% wt-%, more typically >90 wt-% and most typically >95 wt-% of water. The liquid phase can additionally comprise other liquids, typically organic solvents, e.g., ethanol. Typically the concentration of such solvents, e.g., ethanol, is <10 wt-%, more typically <3 wt-% and most typically <1 wt-%. In the context of this application the composite disclosed herein is considered a hydrogel since it fulfils the basic criteria of a hydrogel. Accordingly, when referring to the hydrogel composite disclosed herein this referral is equivalent to a referral to the composite.

As used herein, “inhibit” or “inhibiting” refers to the reduction in a sign, symptom or condition (e.g. risk factor) associated with a disease, disorder or condition associated with a tauopathy by an objectively measurable amount or degree compared to a control. In one embodiment, inhibit or inhibiting refers to the reduction by at least a statistically significant amount compared to a control (or control subject). In one embodiment, inhibit or inhibiting refers to a reduction by at least 5 percent compared to control (or control subject). In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control (or control subject).

Injectable means, in the context of this application, administrable via a surgical administration apparatus, e.g., a needle, a catheter or a combination of these. Shear-thinning in the context of this application is a rheological property of a composition. Whenever the shear stress or shear rate of such a composition is altered, the composition will gradually move towards its new equilibrium state and at lower shear rates the shear thinning composition is more viscous, and at higher shear rates it is less viscous. Thus shear-thinning refers to an effect where a fluid's viscosity, i.e., the measure of a fluid's resistance to flow, decreases with an increasing rate of shear stress.

Injectable Gel or Hydrogel in a context of this application is a rheological property of a composition. Before injection, e.g., as stored in a syringe and/or in an aluminum foil at temperatures <37° C., e.g., at room temperature (at 20-25° C.), or at refrigerator temperatures (at 3-6° C.) the composition is a gel, i.e., the elastic modulus (measured under small angle oscillatory shear) G′ is greater than the viscous modulus G″ and the loss factor, tan δ=(G″/G′), is less than 1. Although the hydrogel composite structure is a gel-like structure and the composite structure remains stable and non-flowing as stored at rest, the gel structure is so loose that it is shear-thinning when shear stress, e.g., in the form of injection through a needle from a syringe is applied, e.g., by using 25G needle (0.50 mm×25 mm).

Non-flowing and structurally stable when stored at rest refers to the stable composite hydrogel structure which is comprised of silica particles in the silica hydrogel. The stability is indicated by rheological measurements under small angle oscillatory shear by the elastic modulus, G′ that is greater than the viscous modulus and the loss factor that is less than 1. When the elastic modulus is greater than the viscous modulus and the loss factor is less than 1, the structure is non-flowing. The non-flowing structure ensures the stability of the composite hydrogel structure by preventing the phase separation of the silica particles. In other words, the silica particles are embedded in the silica hydrogel and they do not, e.g., precipitate or separate on the bottom of a vessel, e.g., a syringe, where the hydrogel composite is stored, typically at temperatures ≤25° C. Although the composite hydrogel structure is non-flowing as stored at rest, e.g., in a prefilled, ready-to-use syringe, the structure is so loose that it is shear-thinning, and hence injectable through thin needles, as shear stress is applied on the hydrogel composite by injection.

As used herein the term “pharmaceutically acceptable carrier” refers to a diluent, adjuvant, excipient, or vehicle with which a compound/drug product/composition (including a medicament) is administered or formulated for administration. Non-limiting examples of such pharmaceutically acceptable carriers include liquids, such as water, saline, oils and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the term “pharmaceutically acceptable salt” refers to a salt of a therapeutically active compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66: 1-19 (1977)). Certain specific compounds of the present application may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

The term silica refers to amorphous SiO₂ that can be prepared by a sol-gel process. The sol-gel derived silica refers to silica prepared by the sol-gel process wherein the silica is prepared from liquid phase precursors, such as alkoxides, alkylalkoxides, aminoalkoxides or inorganic silicate solutions, which by hydrolysis and condensation reactions form a sol that turns to a gel or forms a stable sol. The liquids in the stable silica sol can be evaporated, which results in the formation of a powder consisting typically of colloidal silica particles. The resulting gels/particles can be optionally aged, dried and heat-treated and if heat-treated, preferably below 700° C. The sol-gel derived silica prepared below 700° C. is commonly amorphous. The heat treatment is typically skipped if the gel contains a biologically active agent, such as drugs and active pharmaceutical ingredients (e.g., an oligopeptide or a peptidomimetic as described herein). The formed gel is then typically only aged (typically at ≤40° C.) and dried (typically at ≤40° C.). The sols can be let to gel in a mould for form-giving. The sol-gel derived silica can also be prepared by processing to different morphologies by simultaneous gelling, aging, drying and form-giving, e.g., by spray-drying to microparticles, by dip/drain/spin-coating to films, by extrusion to monolithic structures or by spinning to fibres.

The term silica sol refers to a suspension, i.e., mixture of a liquid (the continuous phase) and a solid phase (the dispersed phase), where the solid phase is comprised of silica particles and/or aggregated silica particles, where the particle size of the silica particles and/or aggregates is typically below 1 μm, i.e., the silica particles and/or particle aggregates are colloidal. A silica sol is commonly prepared from alkoxides or inorganic silicates that, via hydrolysis, form either partly hydrolysed silica species or fully hydrolysed silicic acid. The liquid phase is typically comprised of water and hydrolysis and condensation products, such as ethanol. Subsequent condensation reactions of SiOH-containing species lead to formation of larger silica species having an increasing amount of siloxane bonds. These species form nanosized, colloidal particles and/or particle aggregates. Depending on the conditions, the silica sol remains as a stable colloidal suspension or it turns into a gel.

The sol should be understood to be a homogeneous mixture of at least one liquid phase and one solid phase, i.e., a colloidal dispersion, where the liquid phase(s), e.g., water, ethanol and residuals of silica precursors, is the continuous phase and the solid phase(s), e.g., colloidal particles of silica and/or as partly or fully hydrolysed silica and/or aggregates of said particles are homogeneously dispersed in the said liquid phase characterized in that the sol has clear flow properties and the liquid phase is dominating. A suspension can also be called a sol especially if the solid particles are colloidal, being smaller than 1 μm in diameter. In the context of the present application, however, the term sol refers to a colloidal dispersion wherein the solid particles are <50 nm and the term suspension refers to a dispersion wherein the solid particles are >50 nm.

As used herein, the terms “subject,” “individual,” or “patient” can be an individual organism, a vertebrate, a mammal, or a human. Subjects include, but are not limited to, rats, mice, chicken, cows, monkeys, rabbits, primates and the like, prior to testing in human subjects. The subject can be human.

As used herein, the terms “treating” or “treatment” refer to therapeutic treatment, wherein the object is to reduce, alleviate or slow down (lessen) a pre-existing disease, disorder or condition or its related signs, symptoms or conditions. By way of example, but not by way of limitation, a subject is successfully “treated” for a disease if, after receiving an effective amount of the compound/composition/drug product or a pharmaceutically acceptable salt thereof, the subject shows observable and/or measurable reduction in or absence of one or more signs, symptoms or conditions associated with the disease, disorder or condition

### Pharmaceutical Compositions

In certain embodiments, the present application is directed to a pharmaceutical composition. In certain embodiments, the pharmaceutical composition comprises the depot formulations as disclosed herein. In some embodiments, the depot formulation further comprises an additional therapeutic compound (i.e., agent) and a pharmaceutically acceptable carrier. At least one additional therapeutic agent can refer to an agent useful in the treatment of a disease, such as Leber's Hereditary Optic Neuropathy. The pharmaceutical composition can be a medicament.

Pharmaceutical compositions can be prepared by combining a compound, e.g., an oligopeptide or a peptidomimetic compound with a pharmaceutically acceptable carrier and, optionally, one or more additional therapeutic agents.

An “effective amount” refers to any amount that is sufficient to achieve a desired biological effect (e.g., an amount that treats, inhibits, ameliorates, or delays the onset of the disease, disorder or condition, or the physiological signs, symptoms or conditions of the disease or disorder). Combined with the teachings provided herein, by choosing among the various active compounds and weighing factors such as potency, relative bioavailability, subject body weight, severity of adverse side-effects and mode of administration, an effective prophylactic (i.e., preventative) or therapeutic treatment regimen can be planned which does not cause substantial unwanted toxicity and yet is effective to remedy the condition, disorder or disease of a particular subject. The effective amount for any particular indication can vary depending on such factors as the disease, disorder or condition being treated, the particular compound of the present application being administered, the size of the subject, or the severity of the disease, disorder or condition. The effective amount may be determined during pre-clinical trials and clinical trials by methods familiar to physicians and clinicians. One of ordinary skill in the art can empirically determine the effective amount of a particular compound of the present application and/or other therapeutic agent without necessitating undue experimentation. A maximum dose may be used, that is, the highest safe dose according to some medical judgment. Appropriate systemic levels can be determined by, for example, measurement of the subject's peak or sustained plasma level of the drug. Locally delivered doses can be determined by, for example, the level in a particular compartment of the body, such as the vitreous humor of the eye. “Dose” and “dosage” are used interchangeably herein. A dose can be administered by oneself, by another or by way of a device (e.g., a pump or syringe). The compounds/compositions/drug products can also be administered in combination with one or more additional therapeutic compounds/agents (a so called “co-administration” where, for example, the additional therapeutic agent could be administered simultaneously, sequentially or by separate administration).

Compounds for use in therapy or prevention can be tested in suitable animal model systems. Similarly, for in vivo testing, any of the animal model system known in the art can be used prior to administration to human subjects. Suitable animal model systems include, but are not limited to, rats, mice, chicken, cows, monkeys, rabbits, and the like, prior to testing in human subjects.

A therapeutic compound and optionally other therapeutic agents may be administered in freebase equivalent form or in the form of a pharmaceutically acceptable salt. When used in medicine, the salts should be pharmaceutically acceptable, but non-pharmaceutically acceptable salts may conveniently be used to prepare pharmaceutically acceptable salts thereof. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound. As another example, the salt may comprise less than one inorganic or organic acid molecule per molecule of base, such as two molecules of compound per molecule of tartaric acid.

Pharmaceutical compositions of the present application contain an effective amount of a therapeutic compound as described herein (e.g. elamipretide, Compound I, Compound II, Compound III or Compound IV) and may optionally be disbursed in a pharmaceutically acceptable carrier. The components of the pharmaceutical compositions also are capable of being commingled with the compounds of the present application, and with each other, in a manner such that there is no interaction which would substantially impair the desired pharmaceutical efficiency.

Dosage, toxicity and therapeutic efficacy of any therapeutic compounds, compositions (e.g., formulations or medicaments), other therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. A dose can be formulated in animal models to achieve a circulating plasma concentration range that includes the IC50 (i.e., the concentration of the test compound which achieves a half-maximal inhibition of symptoms) e. Such information can be used to determine useful doses in humans accurately. Levels of the active pharmaceutical ingredient/therapeutic compound (e.g., the oligopeptide or the peptidomimetic) in plasma of an animal or human may be measured, for example, by high performance liquid chromatography.

An exemplary treatment regime can entail administration once a week to once a month, or once a month to once a year, or once a month to once every six months, or once a month to once every three months, once every three months to once every six months, once every six months to once nine months, or once every nine months to once per year. In some embodiments, the treatment regime is an intravitreal injection of the depot formulation about once every three months, once every six months, once every nine months or once about every year. In therapeutic applications, a relatively high dosage at relatively short intervals is sometimes required until progression of the disease, disorder or condition is reduced or terminated, or until the subject shows partial or complete amelioration of symptoms of disease, disorder or condition. Thereafter, the subject can be administered a prophylactic regimen.

For use in therapy, an effective amount of the formulations (e.g. depot formulations) disclosed herein can be administered to a subject by any mode that delivers the compound to the desired surface. Administering a pharmaceutical composition may be accomplished by any means known to the skilled artisan. Routes of administration include but are not limited to oral, topical, intranasal, systemic, subcutaneous, intraperitoneal, intradermal, intraocular, ophthalmicly, transmucosal, intravitreal, or intramuscular administration. Administration includes self-administration, the administration by another and administration by a device.

For ophthalmic or intraocular indications, any suitable mode of delivering the therapeutic compounds or pharmaceutical compositions to the eye or regions near the eye can be used. For ophthalmic formulations generally, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983). Nonlimiting examples of pharmaceutical compositions suitable for administration in or near the eye include, but are not limited to, ocular inserts, minitablets, depot formulations, and topical formulations such as eye drops, ointments, and in situ gels. In one embodiment, a contact lens is coated with a pharmaceutical composition comprising a therapeutic compound disclosed herein.

For topical ophthalmic administration, therapeutic compound or pharmaceutical composition may be formulated as solutions, gels, ointments, creams, suspensions, etc. as are well-known in the art. An ocular gel can comprise silica and be a sol gel or hydrogel. Ointments are semisolid dosage forms for external use such as topical use for the eye or skin. In some embodiments, ointments comprise a solid or semisolid hydrocarbon base of melting or softening point close to human core temperature. In some embodiments, an ointment applied to the eye decomposes into small drops, which stay for a longer time period in conjunctival sac, thus increasing bioavailability.

Ocular inserts are solid or semisolid dosage forms without disadvantages of traditional ophthalmic drug forms. They are less susceptible to defense mechanisms like outflow through nasolacrimal duct, show the ability to stay in conjunctival sac for a longer period, and are more stable than conventional dosage forms. They also offer advantages such as accurate dosing of one or more therapeutic compounds, slow release of one or more therapeutic compounds with constant speed and limiting of one or more therapeutic compounds' systemic absorption. In some embodiments, an ocular insert comprises one or more therapeutic compounds as disclosed herein and one or more polymeric materials. The polymeric materials can include, but are not limited to, methylcellulose and its derivatives (e.g., hydroxypropyl methylcellulose (HPMC)), ethylcellulose, polyvinylpyrrolidone (PVP K-90), polyvinyl alcohol, chitosan, carboxymethyl chitosan, gelatin, and various mixtures of the aforementioned polymers. An ocular insert can comprise silica, including in sol gel or hydrogel form.

The ophthalmic or intraocular formulations and medicaments may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

In some embodiments, the viscosity of the ocular formulation comprising one or more therapeutic compounds is increased to improve contact with the cornea and bioavailability in the eye. Viscosity can be increased by the addition of hydrophilic polymers of high molecular weight which do not diffuse through biological membranes and which form three-dimensional networks in the water. Nonlimiting examples of such polymers include polyvinyl alcohol, poloxamers, hyaluronic acid, carbomers, and polysaccharides, cellulose derivatives, gellan gum, and xanthan gum.

In some embodiments, the ocular formulation can be injected into the eye, for example as a sol-gel. In some embodiments, the ocular formulation is a depot formulation such as a controlled release formulation. Such controlled release formulation may comprise particles, such as microparticles or nanoparticles.

A therapeutic compound or other therapeutic agent or mixtures thereof can be formulated in a carrier system. The carrier can be a colloidal system. The colloidal system can be a sol gel or hydrogel. In one embodiment, therapeutic compound or other therapeutic agent or mixtures thereof can be encapsulated in sol gel or hydrogelwhile maintaining integrity of the therapeutic compound or other therapeutic agent or mixtures thereof.

The carrier or colloidal system can be a sol-gel comprising silica nanoparticles or microparticles that comprises or encapsulates the therapeutic agent(s).

In some embodiments, the therapeutic compound or other therapeutic agent or mixtures thereof are prepared with carriers that will protect the therapeutic compound, other therapeutic agent or mixtures thereof against rapid elimination from the body, such as a controlled release formulation, including implants and microencapsulated delivery systems.

The therapeutic compound(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g., a fibrous mesh constructed from several individual pieces of mesh material) or a plurality of individual compositions (e.g., microparticles or nanoparticles) where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient for at least 2 days. In some embodiments, the implant or depot formulation is constructed and arranged to deliver therapeutic or prophylactic levels of the active ingredient for at least 7 days. In some embodiments, the implant or depot formulation is constructed and arranged to deliver therapeutic or prophylactic levels of the active ingredient for at least 14 days. In some embodiments, the implant or depot formulation is constructed and arranged to deliver therapeutic or prophylactic levels of the active ingredient for at least 30 days. In some embodiments, the implant or depot formulation is constructed and arranged to deliver therapeutic or prophylactic levels of the active ingredient for at least 60 days. In some embodiments, the implant or depot formulation is constructed and arranged to deliver therapeutic or prophylactic levels of the active ingredient for at least 90 days. In some embodiments, the implant or depot formulation is constructed and arranged to deliver therapeutic or prophylactic levels of the active ingredient for at least 180 days. In some embodiments, the implant or depot formulation is constructed and arranged to deliver therapeutic or prophylactic levels of the active ingredient for at least one year. In some embodiments, the implant or depot formulation is constructed and arranged to deliver therapeutic or prophylactic levels of the active ingredient for 15-30 days. In some embodiments, the implant or depot formulation is constructed and arranged to deliver therapeutic or prophylactic levels of the active ingredient for 30-60 days. In some embodiments, the implant or depot formulation is constructed and arranged to deliver therapeutic or prophylactic levels of the active ingredient for 60-90 days. In some embodiments, the implant or depot formulation is constructed and arranged to deliver therapeutic or prophylactic levels of the active ingredient for 90-120 days. In some embodiments, the implant or depot formulation is constructed and arranged to deliver therapeutic or prophylactic levels of the active ingredient for 120-180 days. In some embodiments, the long-term sustained release implants or depot formulation are well-known to those of ordinary skill in the art and include some of the release systems described above. In some embodiments, such implants or depot formulation can be administered surgically. In some embodiments, such implants or depot formulation can be administered topically or by injection.

### Sterilization Procedures

In certain embodiments, the depot formulation is a sterilized formulation.

In certain embodiments, the method of making a depot formulation further comprises a step of sterilizing the depot formulation. The sterilization step may be performed in accordance with any of the procedures described herein, including, but not limited to radiation sterilization (sterilization with, e.g., α-rays, β-rays, γ-rays, neutron beams, electron beams, or X-rays), chemical sterilization (with, e.g., ethylene oxide or hydrogen peroxide), or physical sterilization (with, e.g., heat, filtration, or retort canning). In certain embodiments, the method of making a depot formulation further comprises a step of sterilizing the formulation with gamma ray radiation.

In certain embodiments, the method of making a depot formulation comprises a terminal sterilization step wherein the depot formulation is optionally sealed in a container (such as a syringe), and then subjected to a sterilization step. Preferably, sterilization is by irradiation, e.g., gamma irradiation.

A variety of procedures are known in the art for sterilizing a chemical composition such as the depot formulation of the present invention. Sterilization may be accomplished by chemical, physical, or irradiation techniques. Examples of chemical methods include exposure to ethylene oxide or hydrogen peroxide vapor.

Examples of physical methods include sterilization by heat (dry or moist), retort canning, and filtration. The British Pharmacopoeia recommends heating at a minimum of 160° C. for not less than 2 hours, a minimum of 170° C. for not less than 1 hour and a minimum of 180° C. for not less than 30 minutes for effective sterilization. For examples of heat sterilization, see U.S. Pat. No. 6,136,326, which is hereby incorporated by reference. Passing the chemical composition through a membrane can be used to sterilize a composition. For example, the composition is filtered through a small pore filter such as a 0.22 micron filter which comprises material inert to the composition being filtered. In certain instances, the filtration is conducted in a Class 100,000 or better clean room.

Examples of irradiation methods include alpha radiation, beta radiation, gamma irradiation, neutron beam irradiation, electron beam irradiation, microwave irradiation, and irradiation using visible light. One preferred method is gamma ray (γ-ray) irradiation.

There are several sources for electron beam irradiation. The two main groups of electron beam accelerators are: (1) a Dynamitron, which uses an insulated core transformer, and (2) radio frequency (RF) linear accelerators (linacs). The Dynamitron is a particle accelerator (4.5 MeV) designed to impart energy to electrons. The high energy electrons are generated and accelerated by the electrostatic fields of the accelerator electrodes arranged within the length of the glass-insulated beam tube (acceleration tube). These electrons, traveling through an extension of the evacuation beam tube and beam transport (drift pipe) are subjected to a magnet deflection system in order to produce a “scanned” beam, prior to leaving the vacuum enclosure through a beam window. The dose can be adjusted with the control of the percent scan, the beam current, and the conveyor speed. In certain instances, the electron-beam radiation employed may be maintained at an initial fluence of at least about 2 μCurie/cm², at least about 5 μCurie/cm², at least about 8 μCurie/cm², or at least about 10 μCurie/cm². In certain instances, the electron-beam radiation employed has an initial fluence of from about 2 to about 25 μCurie/cm². In certain instances, the electron-beam dosage is from about 5 to 50 kGray, or from about 15 to about 20 kGray with the specific dosage being selected relative to the density of material being subjected to electron-beam radiation as well as the amount of bioburden estimated to be therein. Such factors are well within the skill of the art.

Procedures for sterilization using visible light are described in U.S. Pat. No. 6,579,916, which is hereby incorporated by reference. The visible light for sterilization can be generated using any conventional generator of sufficient power and breadth of wavelength to effect sterilization. Generators are commercially available under the tradename PureBright® in-line sterilization systems from PurePulse Technologies, Inc. 4241 Ponderosa Ave, San Diego, Calif. 92123, USA. The PureBright® in-line sterilization system employs visible light to sterilize clear liquids at an intensity approximately 90000 times greater than surface sunlight. If the amount of UV light penetration is of concern, conventional UV absorbing materials can be used to filter out the UV light.

One method of sterilizing the formulation as provided herein is by radiation sterilization. Such radiation includes α-rays, β-rays, γ-rays, neutron beams, electron beams, and X-rays. In certain preferred embodiments, the radiation is gamma ray (γ-ray) sterilization. Illustrative radiation dosages include the following: from about 0.5-10 MRad (5-100 kGy), from about 1.5-5.0 MRad (10-50 kGy) or about 2-4 MRad (20-40 kGy) of radiation. For example, the γ-ray sterilization or may be carried out at about one of the following dosages, in kGY: 10, 15, 20, 25, 30, 35, 40, 45 or 50. Generally the dose of electron beam or gamma radiation will range from about 10 to 50 kGy or from 20 to 40 kGy.

The dose of radiation may be determined based on the bioburden level (initial contamination) of the composition prior to sterilization. The exposure time will depend upon the nature of the composition, the strength of the beam, and on the conveyor speed, among other variables, and is typically less than one minute; generally in the range of tenths of a second to several seconds. Dosimeters may be used to determine the optimal exposure times of the particular sample or composition being irradiated.

The temperature of the sterilization procedure may range from about 2° to 50° C., or about 15° to 40° C. or about 20° to 30° C. Typically, irradiation is carried out under ambient conditions.

In certain embodiments, the depot formulation is sterilized to provide a Sterility Assurance Level (SAL) of at least 10⁻³, or at least 10⁻⁴, or even at least 10⁻⁵. The Sterility Assurance Level measurement standard is described, for example, in ISO/CD 14937, the entire disclosure of which is incorporated herein by reference. In some instances, a sterile composition as provided herein may possess a Sterility Assurance Level is at least 10⁻⁶ (that is a probability of a nonsterile unit of greater than one in a million).

The depot formulation may be contained in any type of at least partially electron beam or gamma ray permeable container, including, but not limited to, glass, plastic, foil and film-formed packages. The container may be sealed, or alternatively may have an opening. Examples of glass and, in some cases, plastic containers include, but are not limited to, ampules, vials, syringes (single, dual or multiplets), pipettes, applicators, tubes and the like.

For example, the container may be a syringe, or a syringe that includes a syringe cap that is optionally vented. Examples of vented syringe caps include Vented FLL cap 3 micron filter (Qosina, P/N 12089), a Female Luer cap-vented hex luer fit (Qosina, P/N 6570), and a Luer tip syringe cap (Value Plastics (Fort Collins, Colo.), P/N VPM0480201N).

Containers such as but not limited to those described above may also be further packaged and sealed, for example in a pouch such as a sealed foil pouch. Packaging materials for use in the pharmaceutical and medical industry are well known. Containers such as those described in “Pharmaceutical Packaging Technology”, Dean, D. A., et al., Ed., Taylor & Francis, Inc., 2000, as applicable for compositions of the type provided herein are contemplated for use in the present method and for the compositions and kits provided herein.

The penetration of electron beam or gamma irradiation is generally a function of the packaging material selected. In certain embodiments, if it is determined that there is insufficient penetration from the side of a stationary electron beam or gamma ray, the container may be flipped or rotated to achieve adequate penetration. Alternatively, the electron beam or gamma ray source can be moved about a stationary package or container. In order to determine the dose distribution and dose penetration in product load, a dose map can be performed. A dose map may be used to identify the minimum and maximum dose zone for a particular product, package or container. In certain embodiments, after the container containing the depot fomrulation is sterilized, for example, with electron beam or gamma irradiation, the container may be subjected to additional sterilization. For example, the container may be placed in a kit with other components that may require sterilization. In this process, the entire kit may then be sterilized. For example, the entire kit may be further sterilized by chemical (e.g., with ethylene oxide or hydrogen peroxide vapor), physical (e.g., dry heat) or other techniques such as microwave irradiation, electron beam or gamma irradiation.

The invention now being generally described, it will be more readily understood by reference to the following examples which are included merely for purposes of illustration of certain aspects and embodiments of the present invention, and are not intended to limit the invention.

### EXAMPLES

Methods for preparing the formulations in Examples 1 and 2 below were performed essentially as described in WO 2014/207304 and WO 2017/068245 (each incorporated herein by reference for all purposes), but modified as specifically described in each Example, below.

### Example 1: Formulation of Manufactured Silica-API-Silica Hydrogel Composites (Depot) with Elamipretide

### Formulation Specifications:

Total Drug Dose: 270 μg API dose in a 50 μL injection
Daily Drug Dose: 3 μg
In vivo release profile: sustained release for 3 months
Administration Route: Intravitreal injection
Injection volume: less than or equal to 50 μL
Needle size: 30 gauge
Injection frequency: recurring every 3 months.

Information in the Table 1 provides specifics of the formulation prepared and its analysis.
1) Microparticle formulation used to manufacture injectable hydrogel composite
2) Mass of microparticles (g) per 1 mL of hydrogel precursor (R400)
3) Weight-% of microparticles in the depot (500 mg of microparticles per 1 gram of depot)
4) Measured amount of API in the final depot formulation, density of the depot estimated to be 1.2 g/mL
5) pH of the final depot formulation
6) Manual injectability testing was done with 100 μL injection (after priming)

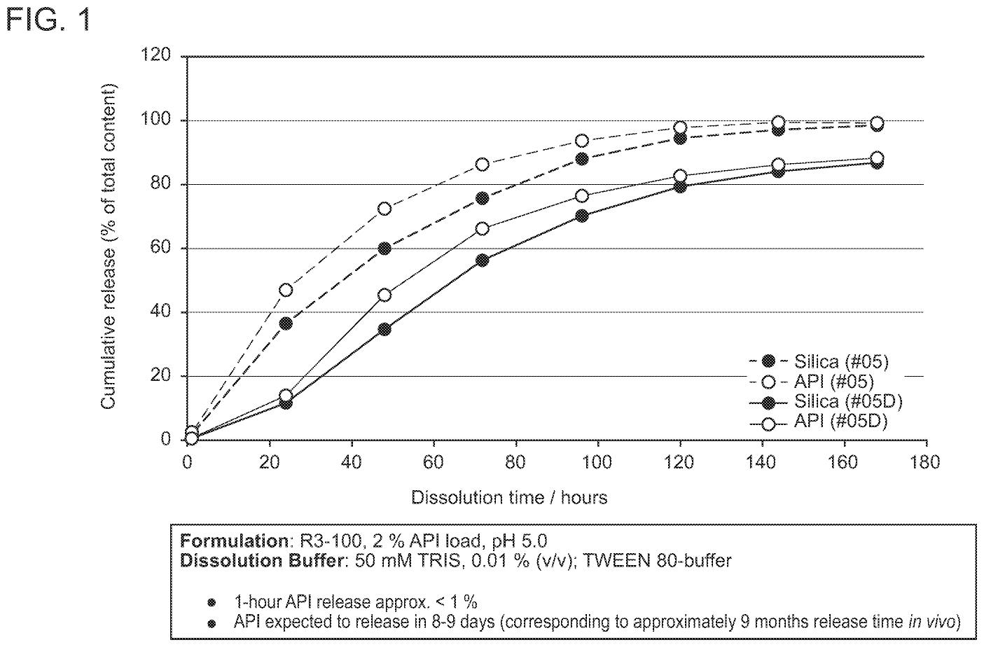

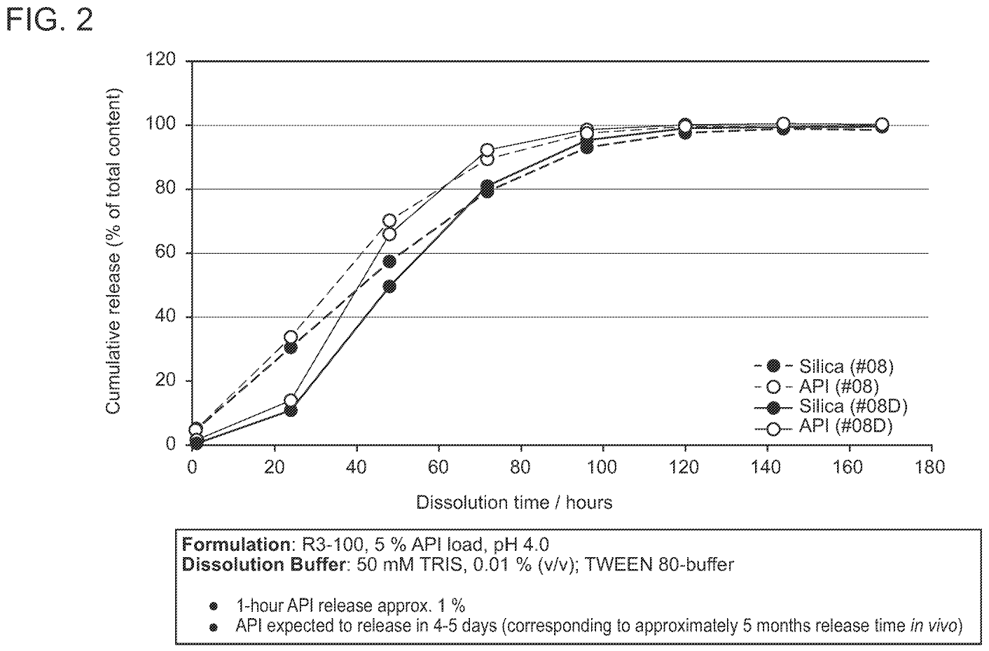

In vitro in sink dissolution data for the composites described in Table 1 are shown in FIG. 1 and FIG. 2.

### Discussion:

FIG. 1 illustrates that formulation #05D released API (i.e., elamipretide) over about 8-9 days and that should correspond to approximately 9 months in vivo.

FIG. 2 illustrates that formulation #08D released API (i.e., elamipretide) over about 4-5 days and that should correspond to approximately 5 months in vivo.

The data in this Example 1 and FIGS. 1 and 2 demonstrates that the release rates of the API can be tailored to the desired release criteria by modulating the conditions under which the particles are formed (e.g., formation pH).

### Example 2: Formulation of Manufactured Silica-API-Silica Hydrogel Composites (Depot) with Compound I

1) Microparticle formulation used to manufacture injectable hydrogel composite
2) Mass of microparticles (g) per 1 mL of hydrogel precursor (R400)
3) Weight-% of microparticles in the depot (500 mg of microparticles per 1 gram of depot)
4) Measured amount of API in the final depot formulation, density of the depot estimated to be 1.2 g/mL
5) pH of the final depot formulation
6) Manual injectability testing was done with 100 μL injection (after priming)

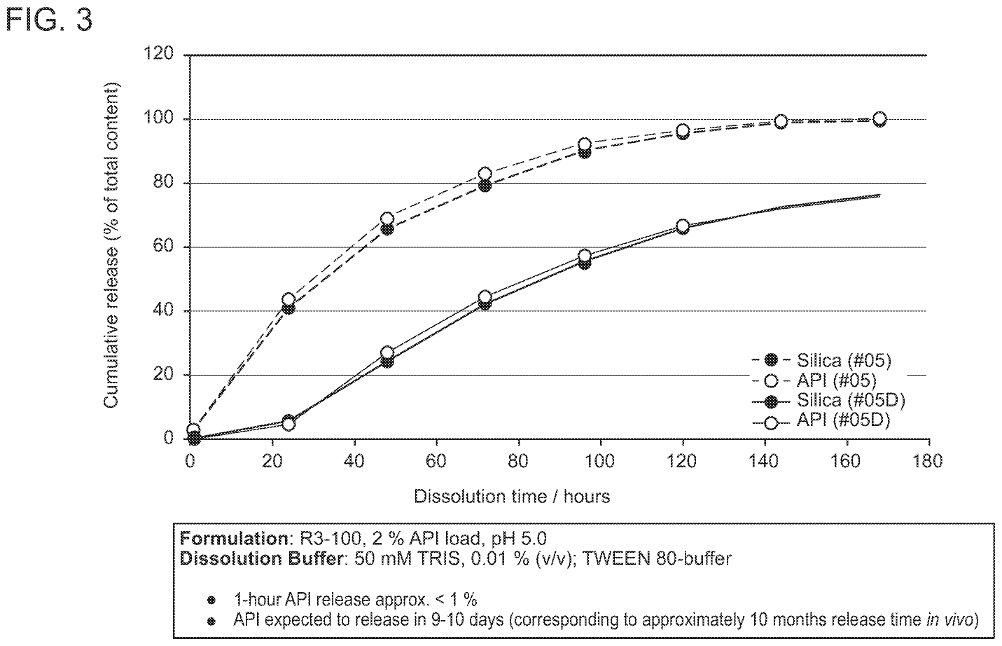

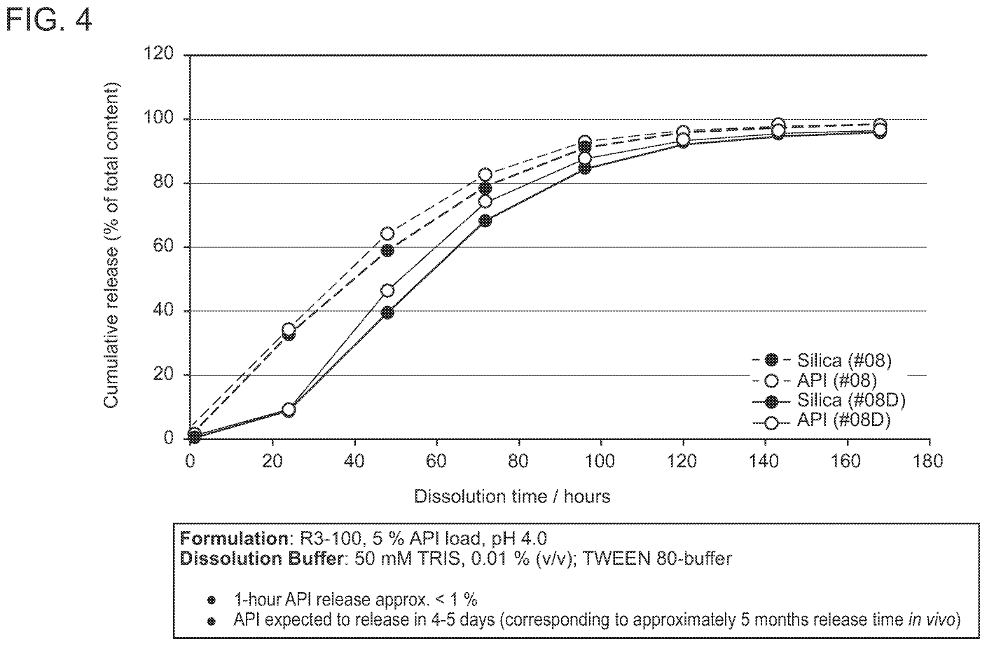

In vitro in sink dissolution data for the composites described in Table 2 are shown in FIG. 3 and FIG. 4.

### Summary of Results

Hydrogel component decreases both silica and API release rates of formulations #05D.
Hydrogel component did not have significant effect on release rates of formulations #08D compared to #05D.
All depot formulations injected through either a 25 gauge or 27 gauge size needle.
Manufacturing of the hydrogel composite was easy.
Microparticle wetting was fast, and workability of the suspension was good.
Data for Compound I formulations #05D and #08D were similar to what was observed for elamipretide, specifically:
   FIG. 3 illustrates that formulation #05D released API (i.e., Compound I) over about 9-10 days and that should correspond to approximately 10 months in vivo.
   FIG. 4 illustrates that formulation #08D released API (i.e., Compound I) over about 4-5 days and that should correspond to approximately 5 months in vivo.
The data in this Example 2 demonstrates that the release rates of the API can be tailored to the desired release criteria by modulating the conditions under which the particles are formed (e.g., formation pH).

TABLE 1
Silica-hydrogel composites with elamipretide.

| Depot | MP | | | Micro- | | | |
|---|---|---|---|---|---|---|---|
| Formu- | formu- | | | particle | Amount | | |
| lation | lation | | MP | amount | of | | Injecta- |
| code | code | Hydrogel | added | (wt-%) | API | pH | bility * |
| #05D | #05 | R400 | 1 g/mL | 50% | 320 μg/ | 6.4 | 25 G |
| | | | | | 50 μL | | |
| #08D | #08 | R400 | 1 g/mL | 50% | 870 μg/ | 6.0 | 27 G |
| | | | | | 50 μL | | |

* Needle gauge used to deliver formulation

TABLE 2
Silica-hydrogel composites with Compound I.

| Depot | MP | | | Micro- | | | |
|---|---|---|---|---|---|---|---|
| Formu- | formu- | | | particle | Amount | | |
| lation | lation | | MP | amount | of | | Injecta- |
| code | code | Hydrogel | added | (wt-%) | API | pH | bility * |
| #05D | #05 | R400 | 1 g/mL | 50% | 320 μg/ | 6.4 | 25 G |
| | | | | | 50 μL | | |
| #08D | #08 | R400 | 1 g/mL | 50% | 870 μg/ | 6.5 | 27 G |
| | | | | | 50 μL | | |

* Needle gauge used to deliver formulation

### Example 3: Release of Compound I and Elamipretide in Ammonium Bifluoride

0.1 M NH₄HF₂ is added to silica microparticles to a particleconcentration of 1 mg/mL (i.e., 10 mg of particles to 10 mL of solution).
API is spiked to a concentration of 16 μg/mL or 40 μg/mL with silica-placebo microparticles.
Silica microparticles are dissolved at room temperature
Excess silica-placebo microparticles is added (16 mg) to inactive non- reacted NH₄HF₂
Blank sample→Same formulation without API.
1.5 mL of sample solution is centrifuged at 14000 rpm for 10 mins.
Supernatant is analyzed with stability-indicating RP-HPLC method.
Control/standard→15 μg/mL or 40 μg/mL API in water.

TABLE 3
Stability Results of Compound I Formulation #5.

| | Recovery | Purity |
|---|---|---|
| Sample: Compound I formulation #5 | % | % |
| Control/standard: 16 μg/mL Compound I in H₂O | NA | 99.1 |
| Spiked: 16 μg/mL Compound I in 0.1M NH₄HF₂ | 99.4 | 99.2 |
| Dissolved: formulation #5 in 0.1M NH₄HF₂ | NA | 99.2 |

TABLE 4
Stability Results of Compound I Formulation #8.

| | Recovery | Purity |
|---|---|---|
| Sample: Compound I formulation #8 | % | % |
| Control/standard: 40 μg/mL Compound I in H₂O | NA | 98.4 |
| Spiked: 40 μg/mL Compound I in 0.1M NH₄HF₂ | 100.0 | 98.6 |
| Dissolved: formulation #8 in 0.1M NH₄HF₂ | NA | 98.1 |

### Conclusion:

Relative amount of impurities in fresh API samples and in samples with APIs released from microparticle formulations were on the same level.
Both elamipretide and Compound I were not significantly modified by the formulation process, such that formulation would negatively impact the process as being unsuitable for development of these formulation for controlled release of the APIs tested.

### Example 5: Preparation of the Sol for Microparticles and Preparation of Microparticles

An overview of the silica-API microparticlE-silica hydrogel depot manufacturing process is shown in FIG. 5.

Silica sols for the manufacturing of spray-dried microparticles may be produced by hydrolysis of tetraethyl orthosilicate (TEOS) in water using hydrochloric acid (HCl) as a catalyst at pH 2. The molar ratio of water: TEOS determines the first R-value (1^{st} R) of the microparticle formulation. The active pharmaceutical ingredient (API) may be dissolved into diluent (ethanol or water, or their mixture), and this solution may be then combined with the sol. As a result, the TEOS in the formed solution becomes further diluted, and this is indicated as the second R-value (2^{nd} R), for which the diluent is calculated as if it were water. Prior to spray-drying, the pH of the sol may be adjusted, then the sol may be spray-dried to form microparticles. These microparticles mainly determine the properties of the formulation. A specific experimental procedure is described below.

The silica sol for generating the microparticles was produced by hydrolysis of tetraethyl orthosilicate (TEOS) in water as follows: First, TEOS was mixed with water; the initial molar ratio of water to TEOS (=the first R in the standard nomenclature used to describe the formulation recipe e.g., R10-100) varied from 3 to 10. TEOS is immiscible in water, so it was a two-phase system before hydrolysis. Then, the water-TEOS mixture was adjusted to pH 2 with 0.1 M HCl and became a homogeneous aqueous solution. The amount of water in the HCl is considered when calculating the R-value. After the exothermic hydrolysis reaction, the silica sols were cooled in an ice bath. A scheme showing the molecular polymerization is shown in FIG. 6.

Subsequently, the cold silica sol was added to the API-water solution, indicated as a dilution factor R, i.e., the molar ratio of water to TEOS after dilution (=the second R in the standard nomenclature used to describe the formulation recipe e.g., R10-100). The-second R-value was 100 with all manufactured microparticles.

The impact of the API load-% was also investigated and the API load-% varied from 2% to 5% in relation to the silica content in the sol. Lastly, the sols were adjusted to a final pH with a 0.1 M NaOH solution, and the pH varied from 4.0 to 5.9 before spray-drying. In general, the pH of the sol prior to spray-drying influences the condensation degree of silica and thereby the dissolution rate of the microparticles.

Finally, silica-API microparticles were prepared by spray-drying the sol containing the API with filtered ambient air. The spray-drying parameters are listed in below.

### Spray-Drying Conditions With Büchi Model B-290:

| Inlet | Outlet | | | | |
|---|---|---|---|---|---|
| temper- | temper- | | | Atomization | Nozzle |
| ature | ature | Aspirator | Pump | air flow | diameter |
| 100° C. | 56-60° C. | 35 m³/h | 5.6 | 670 l/h | 0.7 mm |
| | | | ml/min | | |

### Example 6: Preparation of Silica-API Microparticl-Silica Hydrogel Depot Formulations

To prepare an injectable depot, silica sol with high water content (R400 sol, water content 98-99%) is also produced by hydrolysis of TEOS in water using HCl as a catalyst at pH 2. The spray-dried silica-API microparticles, possessing the 1^{st} and 2^{nd} R-values, are mixed with the R400 sol. This silica microparticle—silica hydrogel suspension is used to fill syringes. Syringes may be kept homogenous by rotating them in a rotating mixer until gel is formed. A specific experimental procedure is described below.

Silica sol with high water content (R400, water content 98-99%) was produced by hydrolysis of TEOS and deionized water using 0.1 M HCl as a catalyst. After the hydrolysis, the pH of the sol (i.e., the sol for the hydrogel) was adjusted with 0.1 M NaOH solution to ca. pH 6.

The spray-dried silica-API microparticles from Example 5 were mixed manually with the R400 sol in 1:1 ratio (w:v). The suspension of silica sol and microparticles were mixed by pipetting the suspension up and down, and the batch sizes were ca. 1-3 ml. The suspension was transferred into plastic syringes (1 ml disposable BD syringes) by directly drawing in the suspension with the syringe (without needle), and any possible air bubbles were removed by tapping the syringe. Then, the silica-API microparticle—silica hydrogel suspension was kept homogenous by gently mixing the capped syringes in a tube rotator to prevent particle sedimentation from occurring while the suspension was allowed to gel at ambient temperature for 1-2 days.

### Example 7: In Vitro Dissolution Tests

The properties of the formulations are mainly determined by the microparticles. The 1^{st} R-value determines the silica dissolution rate. In general, the smaller the 1^{st} R is, the faster silica matrix dissolves in water. On the other hand, the 2^{nd} R-value impacts the efficiency of encapsulation. The reported pH is the pH of the sol before spray-drying. pH has an effect on the condensation degree of silica, and thereby on the dissolution rate, but its effect depends on the formulation and the API. The API load-% is the amount of API in the formulation in relation to silica content. The API load-% has an effect on silica structure and dissolution rate. Diluent is the solvent/diluent used for dissolving the API before adding the API in the sol. The diluent may improve solubility of the API and prevent its precipitation, e.g. in the spray-drying. Inlet temperature (IT) in spray-drying plays a role in particle formation by controlling the evaporation rate of the solvent(s).

In vitro dissolution of silica and the release of the API were measured in 50 mM TRIS with 0.01% (v/v) TWEEN® 80 pH 7.4 at 37° C. (in-house referred as TW). The size of the microparticle or hydrogel sample analyzed was ca. 10-30 mg, and the dissolution studies were conducted up to 168 hours (7 days) in a shaking water bath (60 strokes/min). Silica and the API concentrations were kept at in sink condition (free dissolution of the SiO₂ matrix, i.e., silica is kept below 20% of the saturation concentration) in the dissolution medium. The dissolution medium was changed to fresh medium at every time point to keep the silica concentrations below 30 ppm, i.e., to ensure sink conditions were maintained. At the 1-hour sampling time-point and every 24 hours thereafter, the sample was centrifuged before changing the dissolution medium. Three replicate samples were collected at each timepoint, and mean values are shown in the results.

The total silica content of the samples was measured separately by dissolving the samples (10-25 mg) in 0.5 M NaOH solution and dissolution was continued until the cumulative amount of silica dissolved was equal to the total silica content. The total Compound I content was measured by dissolving the samples (10 mg of microparticles or 20 mg of depot formulation) in 0.1 M ammonium bifluoride while elamipretide (10 mg of microparticles or 20 mg of depot formulation) was dissolved in 150 ml of 50 mM glycine buffer, pH 9.4, supplemented with 0.01% (v/v) TWEEN® 80 (in-house referred as GW). Furthermore, the total dissolution in 0.5 M NaOH solution was also conducted for the in sink dissolution samples after their final sampling timepoint to evaluate how much of the sample remained after the 168-hour time-point.

The encapsulation efficiency of the microparticle formulations, defined as the mass ratio of API to silica calculated after accounting for unencapsulated API, were measured in ethanol. The mass of the sample analyzed was ca. 20-30 mg to which ethanol solution was added in a way that the final mass concentration of microparticles in ethanol was 1 mg/ml. The ethanol dissolution studies were conducted up to 24 hours at room temperature. The rationale herein is that the silica matrix of the silica-API microparticles is insoluble in ethanol. Therefore, only non-encapsulated API will dissolve in this condition.

It should be noted that the in sink dissolution test is an accelerated dissolution test method, and the API and silica dissolution profiles do not correspond as such with the dissolution characteristics in vivo without an appropriate in vitro-in vivo correlation (IVIVC) factor. For intravitreous injections it has been shown in the past that an IVIVC factor of 30 can be employed to estimate the in vivo dissolution rate of the silica matrix from the in vitro-in sink dissolution data. Meaning, that the dissolution rate of the silica matrix is 30-times slower in vivo compared to the in sink test conditions.

### Example 8: Formulations with Compound I

### I. Silica-API Microparticle Formulations with Compound I

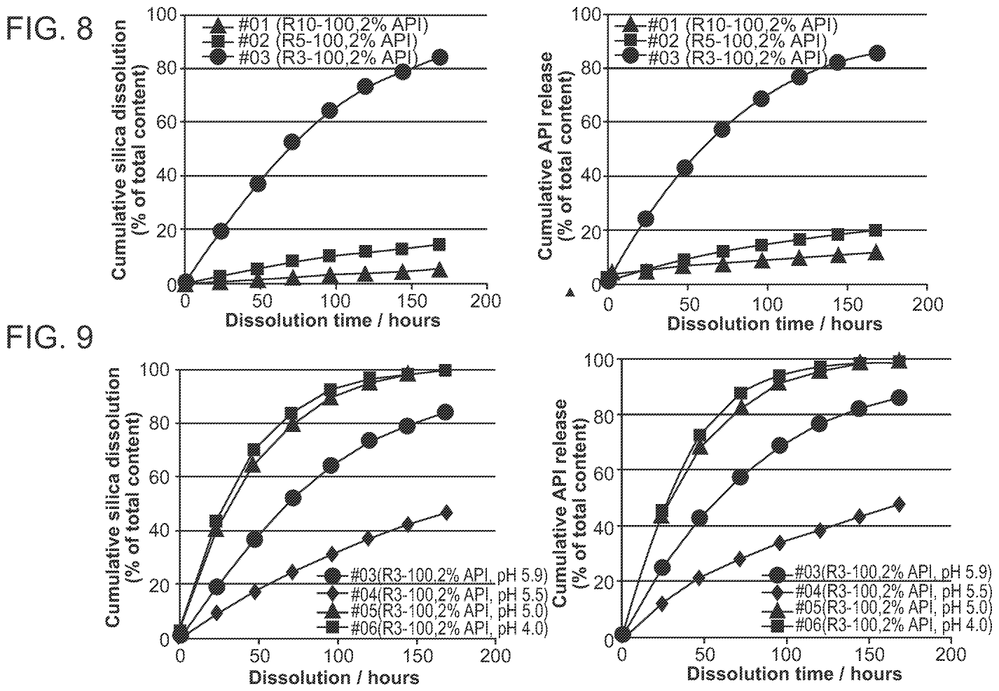

The compositions of the silica-API microparticle formulations manufactured in the study are shown in the table below and the cumulative in sink in vitro release of Compound I and dissolution of the silica matrix for the microparticle formulations are shown in FIGS. 8-10. The figures illustrate the impact of the studied process parameters on the release profiles of the formulations.

Compositions of the manufactured silica-API microparticle formulations and the process parameters studied.

| For- | | | | | | Amount | |
|---|---|---|---|---|---|---|---|
| mula- | | | | API | SiO₂ | of non- | Process |
| tion | 1^{st} and | | Dil- | load- | wt- | encaps. | parameter |
| Code | 2^{nd} R ⁽¹ | pH⁽² | uent ⁽³ | % ⁽⁴ | % ⁽⁵ | API ⁽⁶ | studied |
| #01 | R10-100 | 5.8 | Water | 2% | 86% | <LLOQ | 1^{st} R-value |
| #02 | R5-100 | 5.9 | Water | 2% | 77% | <LLOQ | 1^{st} R-value |
| #03 | R3-100 | 5.9 | Water | 2% | 76% | <LLOQ | 1^{st} R-value |
| #04 | R3-100 | 5.5 | Water | 2% | 84% | <LLOQ | pH |
| #05 | R3-100 | 5.0 | Water | 2% | 84% | <LLOQ | pH |
| #06 | R3-100 | 4.0 | Water | 2% | 79% | <LLOQ | pH |
| #07 | R3-100 | 5.9 | Water | 5% | 82% | <LLOQ | API load-% |
| #08 | R3-100 | 4.0 | Water | 5% | 84% | <LLOQ | API load-% |

⁽¹ R is the molar ratio of water and TEOS (water:TEOS)
⁽²pH of the silica-API sol before spray-drying
⁽³ Initial sol (1^{st} R-value) was diluted to the 2^{nd} R-value with the diluent
⁽⁴ Calculated amount of API in relation to calculated amount of silica
⁽⁵ Total dissolution assay result (0.5M NaOH solution)
⁽⁶ Based on ethanol dissolution analysis (24 hours). Silica matrix does not dissolve in ethanol solution → only non-encapsulated API can be detected

### II. In Vitro in Sink Dissolution of the Silica-API Microparticle Formulations with Compound I

The in vitro dissolution studies were run up to 168 hours (7 days) at in sink conditions. All tested formulations (#01-#08) had low burst release (<5% of the total content), which indicates good encapsulation of the API. This was also verified by ethanol dissolution test which showed that API contents were below the quantitation limit after 24 hours' dissolution.

Summaries of in vitro cumulative dissolution of silica and cumulative release of the API from the silica-API microparticle formulations #01-#03 are shown in FIG. 8. The API load in these microparticle formulations was 2%, and pH was kept constant at 5.9. The 1^{st} R-value of the microparticle formulations had significant effect on the silica dissolution and the API release profiles. Decrease in 1^{st} R-value led to faster dissolution rate of the silica matrix since more hydroxyl groups remain in the silica matrix. Within 7 days, approx. 5% of the silica matrix was dissolved from the slowest formulation (#01) and approx. 84% from the fastest formulation (#03). The API release profile correlates with the silica dissolution profile and 12-87% of the total API content was released within 7 days.

Summaries of in vitro cumulative dissolution of silica and cumulative release of the API from the silica-API microparticle formulations #03-#06 with varying pHs are shown in FIG. 9. The API load in these microparticle formulations was 2%, 1^{st} R-value was kept constant at 3 and 2^{nd} R-value at 100.

In this study, significant differences in the release rates of the API and silica matrix dissolution are seen between formulations #03 and #04 but not between #05 and #06 (FIG. 9). Within 7 days, approx. 47% of the silica matrix was dissolved from the slowest formulation (#04) and 100% from the fastest formulations (#05 and #06). The API release profile correlates with the silica dissolution profile and 48-100% of the total API content was released within 7 days. In vitro in sink dissolution profile of formulation #03 is peculiar since it should have been the slowest formulation due to the highest pH (more condensed silica matrix). The API release correlates with the dissolution of the silica with all studied microparticle formulations.

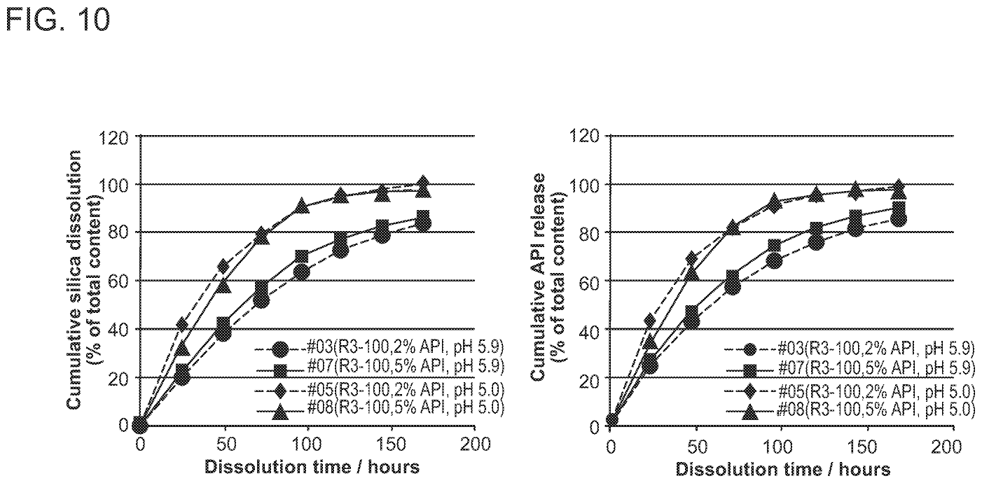

Increasing the API load-% from 2% to 5% was investigated with the R3-100 based formulations (FIG. 10). Formulations #05 and #08 are faster formulations and identical to each other while formulations #03 and #07 are the slower ones. The dissolution results show that increasing the API load from 2% to 5% does not significantly impact the dissolution rate. By contrast, the pH has more significant effect on the dissolution profile.

### III. Particle Size Distribution

The particle size distributions of the developed silica-Compound I microparticle formulations are summarized below.

Particle size distributions of silica-Compound I microparticle formulations. The values represent an average (AVG) of three replicate measurements and the standard deviations (SD).

| Formulation | 1^{st} R and | | | |
|---|---|---|---|---|
| Code | 2^{nd} R -values | D10 (AVG ± SD) | D50 (AVG ± SD) | D90 (AVG ± SD) |
| #01 | R10-100 | 1.37 ± 0.02 μm | 6.69 ± 0.26 μm | 23.46 ± 0.55 μm |
| #02 | R5-100 | 1.33 ± 0.00 μm | 4.60 ± 0.01 μm | 17.33 ± 0.07 μm |
| #03 | R3-100 | 1.37 ± 0.02 μm | 6.69 ± 0.26 μm | 23.46 ± 0.55 μm |
| #04 | R3-100 | 1.12 ± 0.00 μm | 4.02 ± 0.01 μm | 11.14 ± 0.07 μm |
| #05 | R3-100 | 1.13 ± 0.00 μm | 3.40 ± 0.01 μm | 8.46 ± 0.06 μm |
| #06 | R3-100 | 1.74 ± 0.00 μm | 11.68 ± 0.04 μm | 28.32 ± 0.10 μm |
| #07 | R3-100 | 1.07 ± 0.00 μm | 2.63 ± 0.00 μm | 7.47 ± 0.02 μm |
| #08 | R3-100 | 1.17 ± 0.00 μm | 2.57 ± 0.00 μm | 4.96 ± 0.00 μm |

The D-values are the intercepts for 10%, 50% and 90% of the cumulative mass of the sample analyzed. For example, the D10 value is the diameter at which 10% of the sample's mass is comprised of particles with a diameter less or equal than this value. Herein, the particle size of all microparticle formulations is clearly small, since the D50 values are below 10 μm (except #06). Because the silica-API microparticle formulations #05 and #08 showed the most desired characteristics in terms of silica dissolution, API release and particle size distribution, they were chosen as the lead formulations, to be used in the depot formulation development.

### IV. Silica-API Microparticle—Silica Hydrogel Depot Formulations

The compositions of the silica-Compound I microparticle—silica hydrogel (HG) formulations (i.e. silica-Compound I depot formulations) are shown below.

Compositions of the silica-Compound I depot formulations

| | | | Micro- | | | |
|---|---|---|---|---|---|---|
| Formula- | Micro- | Hydrogel | particle | | Amount of | |
| tion | particle | (HG) | amount⁽¹ | Depot | API in 50 μl | |
| Code | Code | formulation | (wt-%) | pH | injection⁽² | |
| #05D | #05 | R400 | 50% | 6.4 | 440 | μg |
| #08D | #08 | R400 | 50% | 6.5 | 1.0 | mg |

¹⁾Wt-% of microparticles in the depot formulation (50 wt-% equals to 500 mg microparticles per 1 gram of depot)
²⁾Measured concentration of API in the final depot formulation in 50 μl injection volume

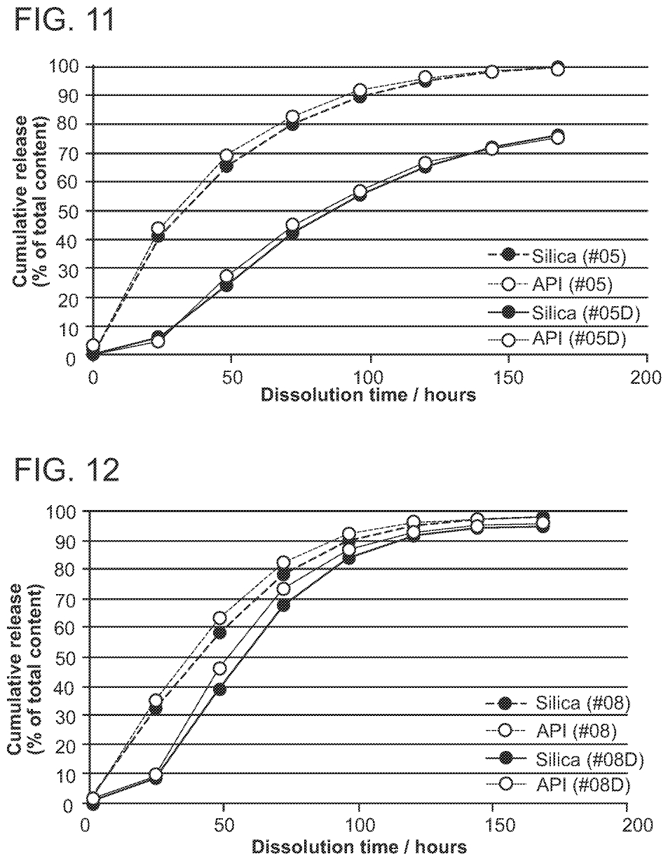

The cumulative in sink dissolution of silica matrix and the API release rates from the depot formulations are shown in FIGS. 11 and 12. The release rates of the corresponding microparticle formulations are also shown in said Figures to indicate the effect of the HG component on the dissolution rate of the microparticle formulations. The cumulative amount of the API released from the (microparticle) formulation #05 within the first 24 hours is decreased from approx. 44% to 5% and (microparticle) formulation #08 from 34% to 10%, when microparticles are incorporated into the silica-HG component. The total release time is increased as 78% (#05D) and 96% (#08D) of the API in the depot formulations is released in 168 hours, whereas both microparticle formulations have released over 96% of the API in 120 hours. This phenomenon is expected since the silica hydrogel surrounding the silica microparticles acts as a barrier and locks-in-place the microparticles. Thus, the depot formulation dissolves as a single solid body rather than permitting the microparticles to disperse into the dissolution buffer. This in turn decreases the reactive surface area of the formulation, which also decreases the dissolution rate.

From FIGS. 11 and 12 it is evident that the HG component decreases at least to some extent the dissolution rate of the microparticle formulation. A short lag-time until 24-h timepoint is observed in the silica dissolution and API release with both depot formulations.

### V. Rheological Measurements and Injectability

The oscillatory measurements were conducted to show the presence of a three-dimensional gel structure within the depot formulation packed into the prefilled syringes. For viscoelastic materials, if the loss factor (tan δ=G″/G′) is greater than 1, the material behaves more like a liquid. On the other hand, if the loss factor is less than 1, the material behaves more like a semisolid, e.g., a gel.

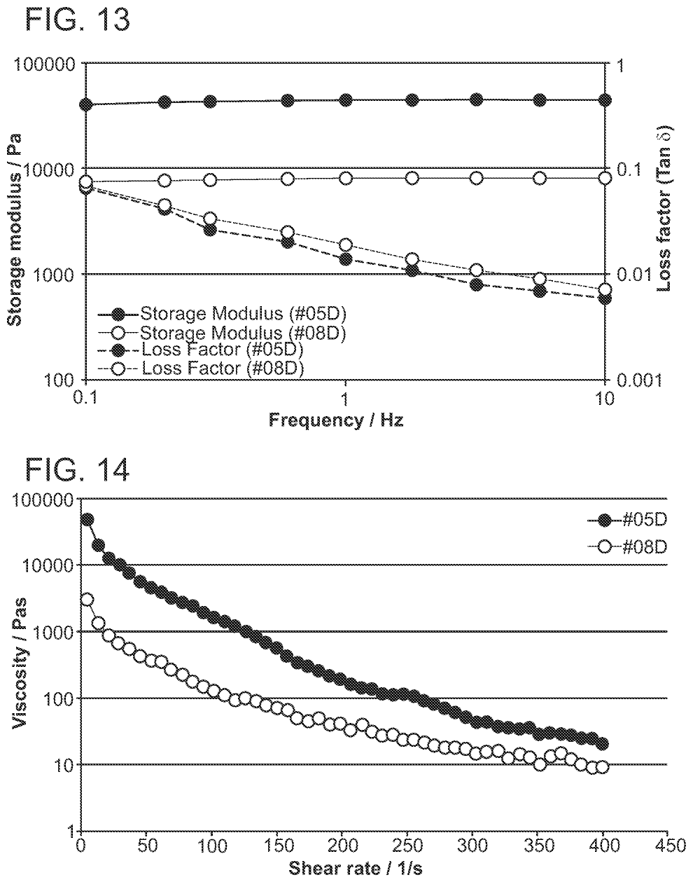

For the depot formulations #05D and #08D, the loss factor is less than 1 in the entire frequency range, shown in FIG. 13. Thus, a three-dimensional gel structure has formed, preventing particle sedimentation within the syringe at rest i.e. during storage. Furthermore, the depot formulation samples elicit adequate storage modulus (G′) values independent of frequency showing good mechanical strength of the depot formulations.

The depot formulations elicit shear-thinning properties since the viscosity decreases as a function of increasing shear rate (FIG. 14). Because the material is solid at rest, the viscosity is high at low shear rates. As the shear rate is increased, the material begins to flow. This shear-thinning characteristic indicates that the material can be injected from the prefilled syringe through thin needles. During the injection procedure the material is subject to shear rates ranging from 10 000 1/s to 100 000 1/s. Essentially the depot formulations are stable solid-like materials at rest and begin to flow as liquids when stressed.

Additionally, manual injectability tests through thin hypodermic needles were performed to show that the shear-thinning phenomenon shown in FIG. 14 is analogous with good injectability of the depot formulations. The results of the injectability tests are shown below.

### Composition of the Depot Formulations, The Number of Injections and the Evaluation of the Injectability.

| Formu- | Micro- | | | Number | | |
|---|---|---|---|---|---|---|
| lation | particle | Fill | Injected | of | Needle | Injection⁽³ |
| Code | wt-%.⁽¹ | volume⁽² | volume | injections | size | evaluation |
| #05D | 50% | 300 μL | 300 μL | 1 | 27G | Easily |
| | | | | | | injected |
| #08D | 50% | 300 μL | 300 μL | 1 | 27G | Easily |
| | | | | | | injected |

⁽¹In relation to the weight of the depot
⁽²Volume of depot filled into the disposable BD 1 mL luer-lock polypropylene syringe
⁽³The samples were successfully injected out through 27G × 1″ (0.4 × 20 mm) hypodermic needles and the force required to inject the material was low (subjectively evaluated).

### Example 9: Formulations with Elamipretide

### I. Silica-API Microparticle Formulations with Elamipretide

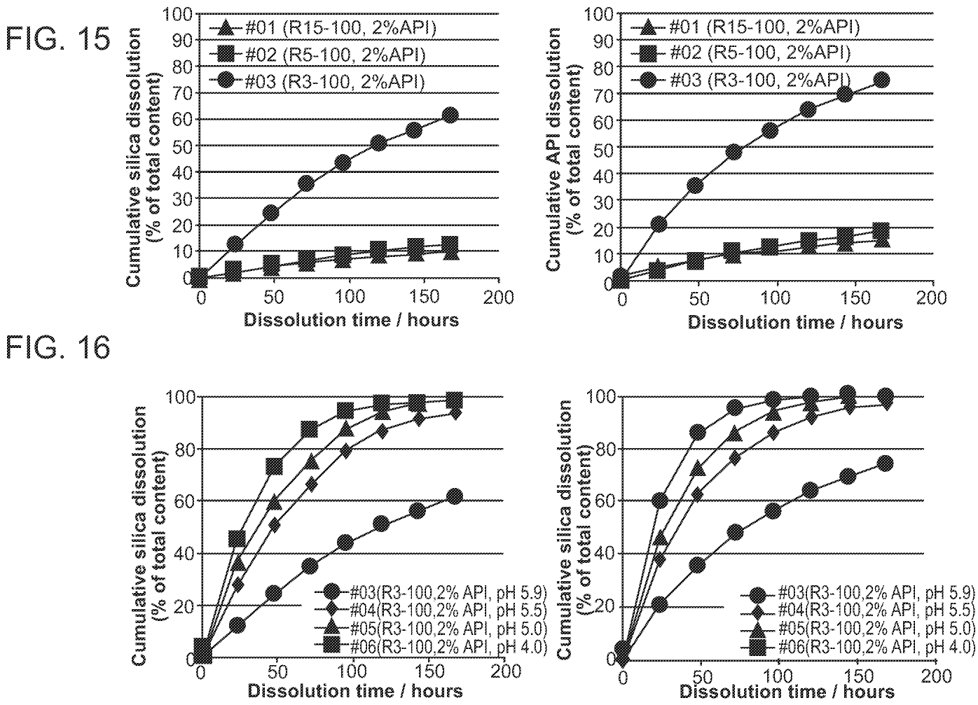

The compositions of the silica-elamipretide microparticle formulations manufactured in the study are shown below and the cumulative in sink release of API for the microparticle formulations are shown in FIGS. 15-17. The figures illustrate the impact of the investigated process parameters on the release profiles of the formulations.

Compositions of the Manufactured Silica-API Microparticle Formulations and the Process Parameters Studied.

| For- | | | | | | Amount | |
|---|---|---|---|---|---|---|---|
| mula- | | | | API | SiO₂ | of non- | Process |
| tion | 1^{st} and | | Dil- | load- | wt- | encaps. | parameter |
| Code | 2^{nd} R ⁽¹ | pH⁽² | uent ⁽³ | % ⁽⁴ | % ⁽⁵ | API ⁽⁶ | studied |
| #01 | R10-100 | 5.8 | Water | 2% | 83% | <LLOQ | 1^{st} R-value |
| #02 | R5-100 | 5.9 | Water | 2% | 84% | <LLOQ | 1^{st} R-value |
| #03 | R3-100 | 5.9 | Water | 2% | 79% | <LLOQ | 1^{st} R-value |
| #04 | R3-100 | 5.5 | Water | 2% | 83% | <LLOQ | pH |
| #05 | R3-100 | 5.0 | Water | 2% | 78% | <LLOQ | pH |
| #06 | R3-100 | 4.0 | Water | 2% | 83% | <LLOQ | pH |
| #07 | R3-100 | 5.9 | Water | 5% | 90% | <LLOQ | API load-% |
| #08 | R3-100 | 4.0 | Water | 5% | 84% | <LLOQ | API load-% |

⁽¹ R is the molar ratio of water and TEOS (water:TEOS)
⁽²pH of the silica-API sol before spray-drying
⁽³ Initial sol (1^{st} R-value) was diluted to the 2^{nd} R-value with the diluent
⁽⁴ Calculated amount of API in relation to calculated amount of silica in the formulation
⁽⁵ Total dissolution assay result (0.5M NaOH solution)
⁽⁶ Based on ethanol dissolution analysis (24 hours). Silica matrix does not dissolve in ethanol solution → only non-encapsulated API can be detected

### II. In Vitro in Sink Dissolution of the Silica-API Microparticle Formulations with Elamipretide

The in vitro dissolution studies were run up to 168 hours (7 days) at in sink conditions. All tested formulations (#01-#08) had low burst release (<5% of the total API content), which indicates good encapsulation of the API. This was also verified by ethanol dissolution test which showed that the API contents were below the detection limit 24 hours' dissolution.

Summaries of in vitro cumulative dissolution of silica and cumulative release of the API from the silica-API microparticle formulations #01-#03 are shown in FIG. 15. The API load in these microparticle formulations was 2%, and pH was kept constant at 5.9. The 1^{st} R-value of the microparticle formulations had an effect on the silica dissolution and the API release profiles. Decrease in 1^{st} R-value led to faster dissolution rate of the silica matrix since more hydroxyl groups remain in the silica matrix. Within 7 days, approx. 10% of the silica matrix was dissolved from the slowest formulations (#01 and #02) and approx. 61% from the fastest formulation (#03). The API release profile correlates with the silica dissolution profile and 15-75% of total API content was released within 7 days.

Summaries of in vitro cumulative dissolution of silica and cumulative release of the API from the silica-API microparticle formulations #03-#06 with varying pHs are shown in FIG. 16. In this study, significant differences in the release rates of the API and silica matrix dissolution are seen between formulations #03 and #06. Approximately 61% of the silica matrix dissolved in 7 days from formulation #03 whereas approx. 100% was dissolved from formulation #06. The API release correlates with the dissolution of the silica with all studied formulations.

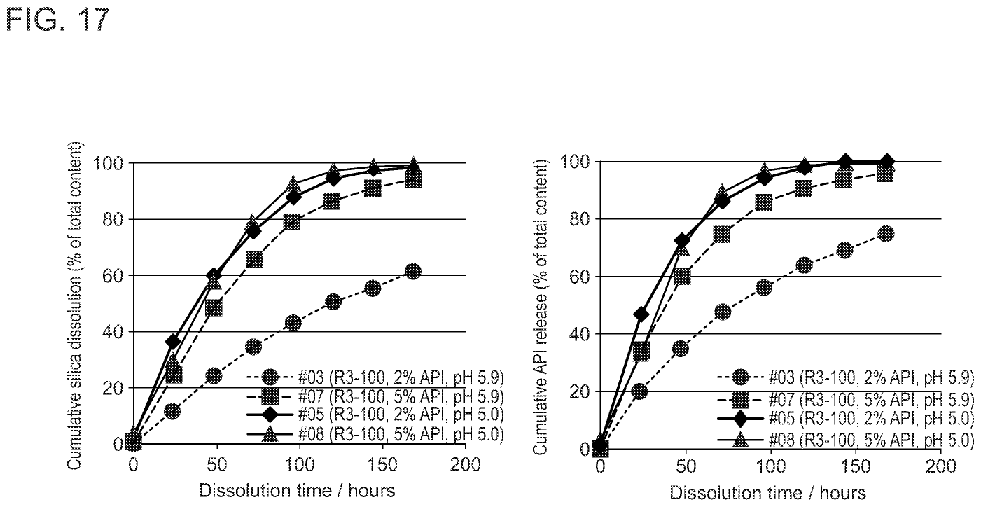

Increasing the API load-% from 2% to 5% was investigated with the R3-100 based formulations (FIG. 17). Formulations #05 and #08 are faster formulations and their dissolution profiles seem to be similar while the difference between formulations #03 and #07 is significant. The dissolution results show that increasing the API load from 2% to 5% does significantly impact the dissolution rate when the pH of the sol prior to spray-drying is higher (pH=5.9). By contrast with lower pH sols, API load does not have significant effect on the dissolution profile.

### III. Particle Size Distribution

The particle size distributions of the developed silica-elamipretide microparticle formulations are summarized below.

Particle size distribution of silica-elamipretide microparticle formulations. The values represent an average (AVG) of three replicate measurements and the standard deviation (SD).

| Formulation | 1^{st} R and | | | | | |
|---|---|---|---|---|---|---|
| Code | 2^{nd} R -values | D10 (AVG ± SD) | D50 (AVG ± SD) | | D90 (AVG ± SD) | |
| #01 | R10-100 | 1.41 ± 0.00 μm | 5.93 ± 0.07 | μm | 22.12 ± 0.38 | μm |
| #02 | R5-100 | 1.22 ± 0.00 μm | 3.65 ± 0.00 | μm | 11.88 ± 0.03 | μm |
| #03 | R3-100 | 1.15 ± 0.00 μm | 3.22 ± 0.00 | μm | 7.40 ± 0.01 | μm |
| #04 | R3-100 | 1.07 ± 0.00 μm | 3.66 ± 0.01 | μm | 9.50 ± 0.09 | μm |
| #05 | R3-100 | 1.17 ± 0.00 μm | 4.74 ± 0.01 | μm | 11.01 ± 0.01 | μm |
| #06 | R3-100 | 2.01 ± 0.01 μm | 15.02 ± 0.13 | μm | 39.03 ± 0.36 | μm |
| #07 | R3-100 | 1.04 ± 0.00 μm | 2.44 ± 0.00 | μm | 5.00 ± 0.03 | μm |
| #08 | R3-100 | 1.32 ± 0.00 μm | 2.63 ± 0.00 | μm | 4.67 ± 0.00 | μm |

The particle size of all microparticle formulations is small, since the D50 values are below 10 μm (except #06). Because the silica-API microparticle formulations #05 and #08 showed the most desired characteristics in terms of silica dissolution, API release and particle size distribution, they were chosen as the lead formulations, to be used in the depot formulation development.

### IV. Silica-API Microparticle—Silica Hydrogel Depot Formulations

The compositions of the silica-elamipretide microparticle—silica hydrogel (HG) formulations (i.e. silica-elamipretide depot formulations) are shown below.

### Compositions of the Silica-Elamipretide Depot Formulations

| | | | Micro- | | | |
|---|---|---|---|---|---|---|
| Formula- | Micro- | | particle | | Amount of | |
| tion | particle | HG | amount⁽¹ | Depot | API in 50 μl | |
| Code | Code | formulation | (wt-%) | pH | injection⁽² | |
| #05D | #05 | R400 | 50% | 6.4 | 440 | μg |
| #08D | #08 | R400 | 50% | 6.0 | 1.0 | mg |

¹⁾Wt-% of microparticles in the depot formulation (50 wt-% equals to 500 mg of microparticles per 1 gram of depot)
²⁾Measured concentration of the API in the final depot formulation in 50 μl injection volume

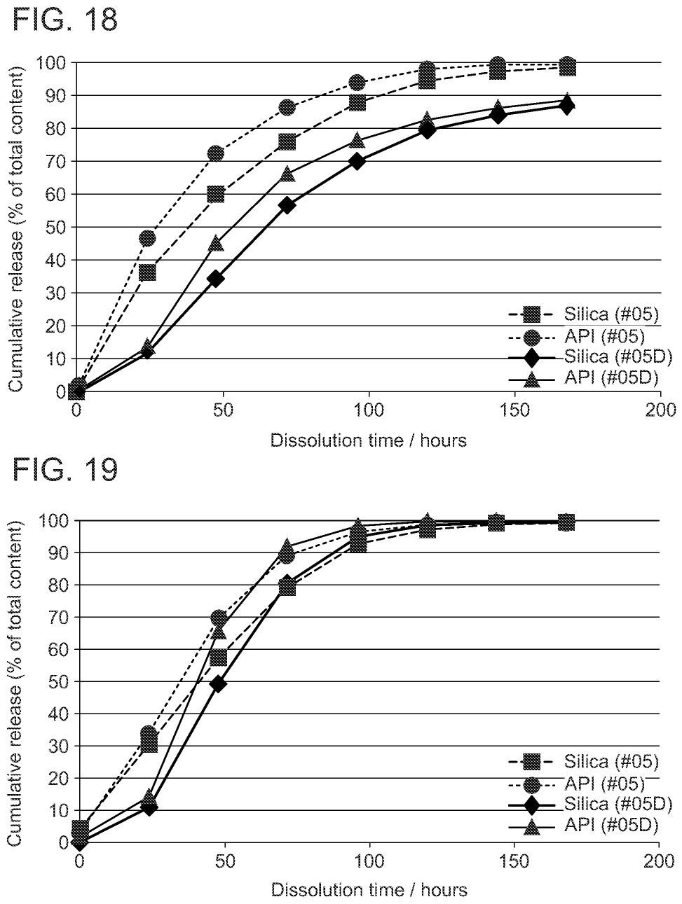

The cumulative in sink dissolution of silica matrix and the API release rates from the depot formulations are shown in FIGS. 18 and 19. The release rate of the corresponding microparticle formulations is also shown in said Figures to indicate the effect of the HG component on the dissolution of the microparticle formulations. The cumulative amount of API released from the microparticle formulation #05 within the first 24 hours is decreased from approx. 47% to 14% and microparticle formulation #08 from 34% to 14%, when microparticles are incorporated into silica HG component. The total release time of formulation #05D is increased as 89% of the API in the depot formulations is released in 168 hours, whereas microparticle formulation has released over 96% of the API in 120 hours. This phenomenon is expected since the silica hydrogel surrounding the silica microparticles acts as a barrier and locks-in-place the microparticles. Thus, the depot formulation dissolves as a single solid body rather than permitting the microparticles to disperse into the dissolution buffer. This in turn decreases the reactive surface area of the formulation, which also decreases the dissolution rate.

From FIGS. 18 and 19 it is evident that the HG component decreases at least to some extent the dissolution rate of the microparticle formulation. A short lag-time until 24-h timepoint is observed in the silica dissolution and API release with both depot formulations.

### V. Rheological Measurements and Injectability

The oscillatory measurements were conducted to show the presence of a three-dimensional gel structure within the depot formulation packed into the prefilled syringes. For viscoelastic materials, if the loss factor (tan δ=G″/G′) is greater than 1, the material behaves more like a liquid. On the other hand, if the loss factor is less than 1, the material behaves more like a semisolid, e.g., a gel.

For the depot formulations #5D and #8D, the loss factor is less than 1 in the entire frequency range, shown in FIG. 16. Thus, a three-dimensional gel structure has formed, preventing particle sedimentation within the syringe at rest i.e., during storage. Furthermore, the depot formulation samples elicit adequate storage modulus (G′) values independent of frequency showing good mechanical strength of the depot formulation.

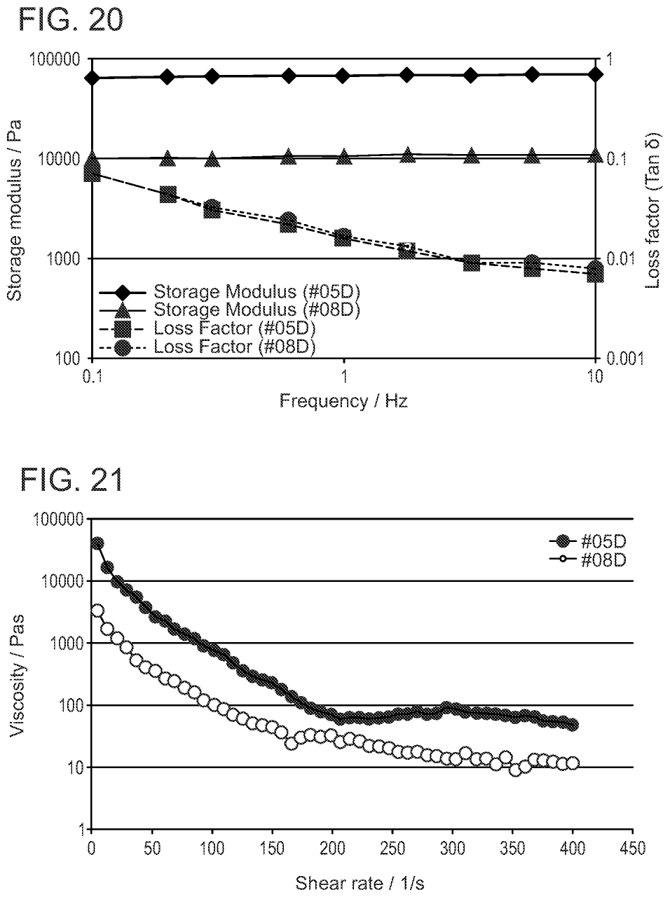

The depot formulations elicit shear-thinning properties since the viscosity decreases as a function of increasing shear rate (FIG. 20). Because the material is solid at rest, the viscosity is high at low shear rates. As the shear rate is increased, the material begins to flow. This shear-thinning characteristic indicates that the material can be injected from the prefilled syringe through thin needles. During the injection procedure the material is subject to shear rates ranging from 10 000 1/s to 100 000 1/s. Essentially the depot formulations are stable solid-like materials at rest and begin to flow as liquids when stressed.

Additionally, manual injectability tests through thin hypodermic needles were performed to show that the shear-thinning phenomenon shown in FIG. 21 is analogous with good injectability of the depot formulations. The results of the injectability tests are shown below.

| Formu- | Micro- | | | Number | | |
|---|---|---|---|---|---|---|
| lation | particle | Fill | Injected | of | Needle | Injection⁽³ |
| Code | wt-%.⁽¹ | volume⁽² | volume | injections | size | evaluation |
| #05D | 50% | 300 μL | 300 μL | 1 | 27G | Easily |
| | | | | | | injected |
| #08D | 50% | 300 μL | 300 μL | 1 | 27G | Easily |
| | | | | | | injected |

⁽¹In relation to the weight of the depot
⁽²Volume of depot filled into the disposable BD 1 mL luer-lock polypropylene syringe
⁽³The samples were successfully injected out through 27G × 1″ (0.4 × 20 mm) hypodermic needles and the force required to inject the material was low (subjectively evaluated).

### Example 10: Manufacture with Continuous Flow Reactor System

A continuous flow reactor system was utilized to scale up the manufacture of Silica-API-Silica Hydrogel Composites (depot) with elamipretide using an API loading of 2% and 5%. A schematic is shown in FIG. 7.

In the continuous flow process, the pH and concentrations of the silica and API remained consistent through the manufacturing process.

### Particle Size Distribution Measurements of Eanufactured Elamipretide Microparticular Bormulation #09 and #10-5 Consistency Batches:

| | | | |
|---|---|---|---|
| Formulation #09 (R3-100, 2% API | 1.32 ± | 3.30 ± | 9.91 ± |
| load, 29 September) | 0.00 μm | 0.00 μm | 0.06 μm |
| Formulation #09 (R3-100, 2% API | 1.21 ± | 2.84 ± | 6.65 ± |
| load, 23 December) | 0.00 μm | 0.00 μm | 0.03 μm |
| Formulation #09 (R3-100, 2% API | 1.17 ± | 2.74 ± | 5.92 ± |
| load, 24 December) | 0.00 μm | 0.00 μm | 0.01 μm |
| Formulation #10-5 (R3-100, 5% | 1.03 ± | 2.46 ± | 5.43 ± |
| API load, 23 September) | 0.00 μm | 0.00 μm | 0.06 μm |
| Formulation #10-5 (R3-100, 5% | 1.04 ± | 2.69 ± | 8.46 ± |
| API load, 21 October) | 0.00 μm | 0.00 μm | 0.03 μm |
| Formulation #10-5 (R3-100, 5% | 1.03 ± | 2.63 ± | 8.08 ± |
| API load, 27 October) | 0.00 μm | 0.00 μm | 0.01 μm |

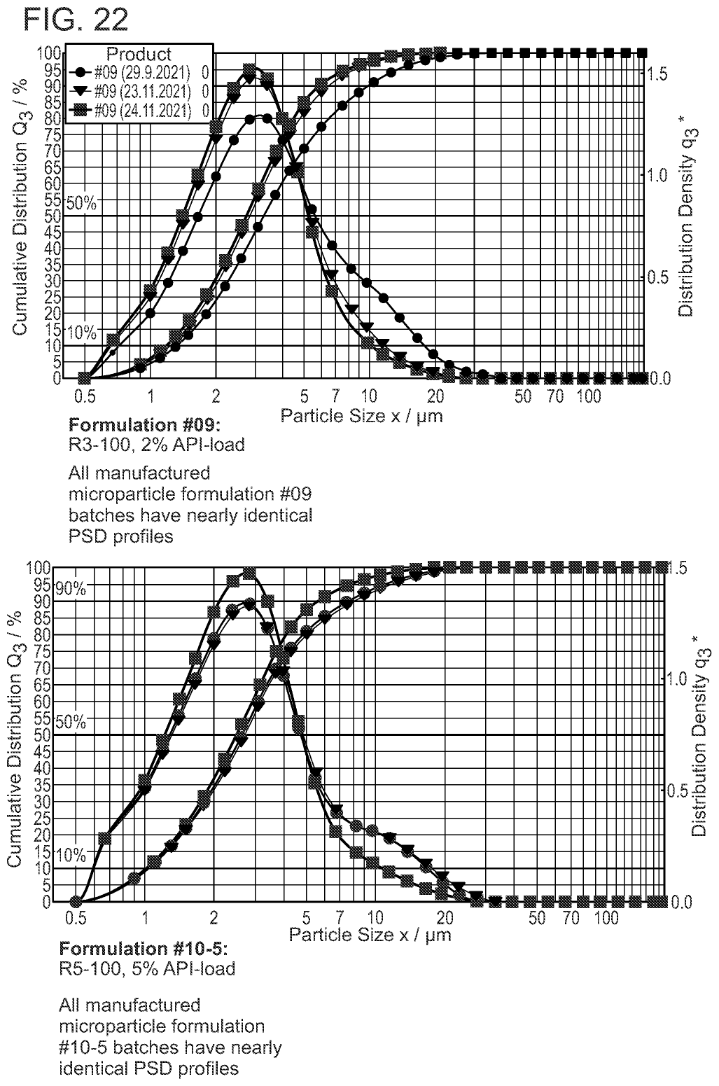

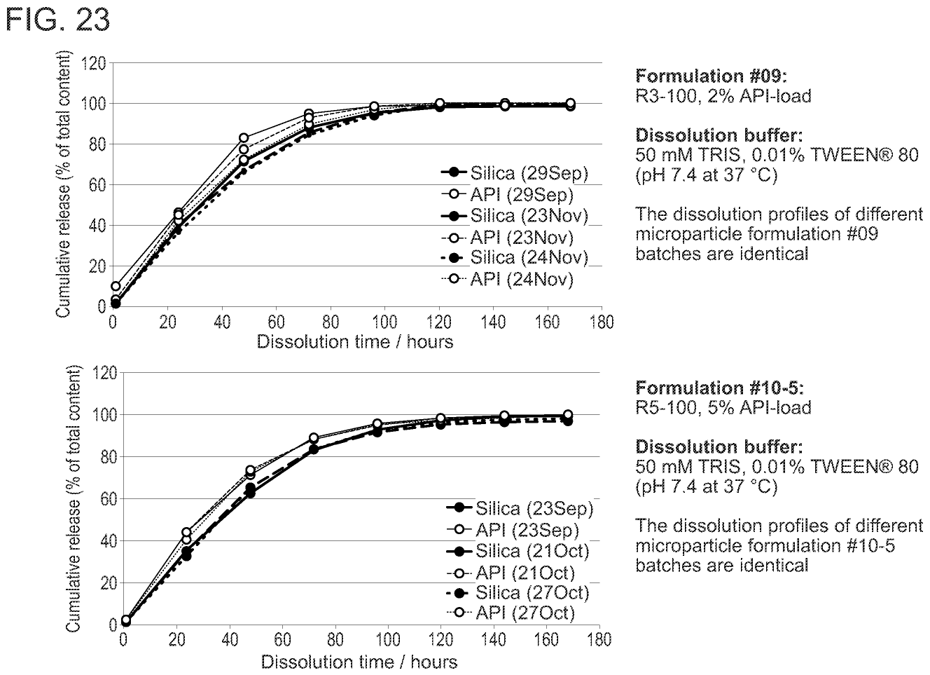

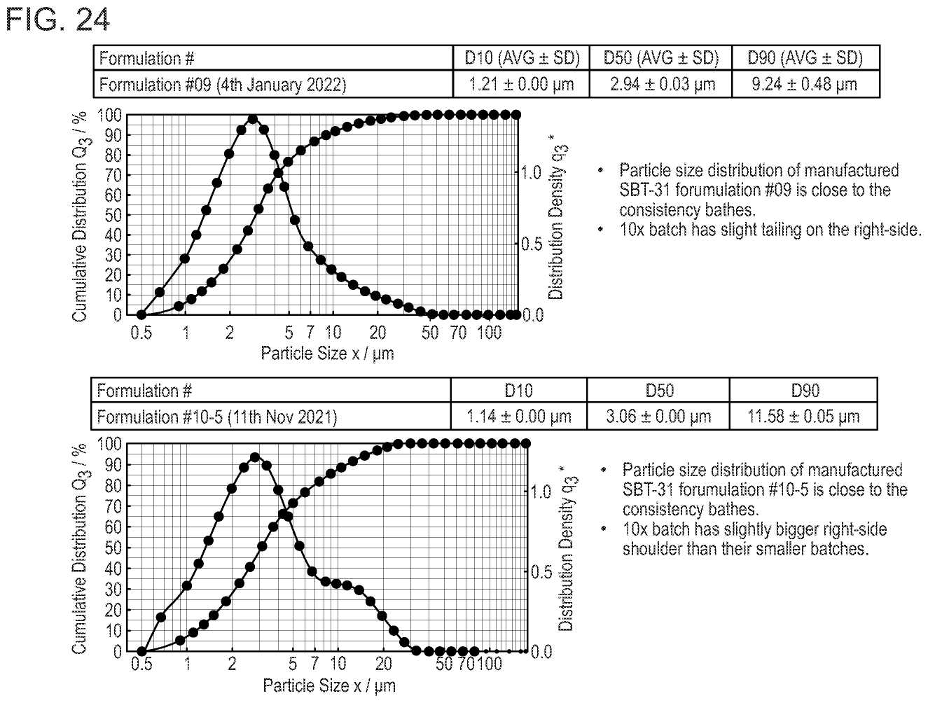

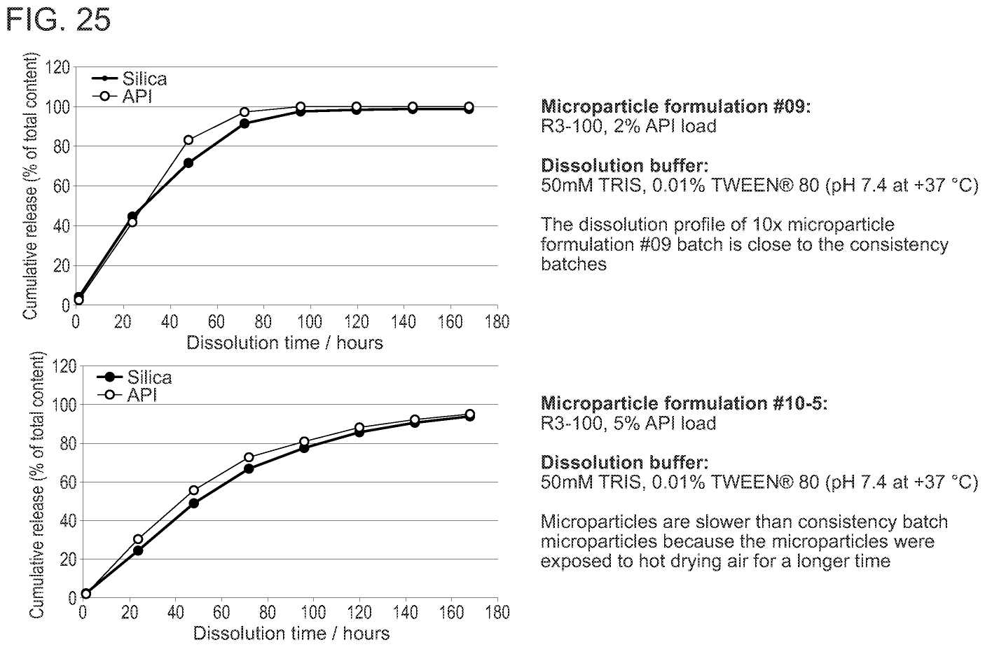

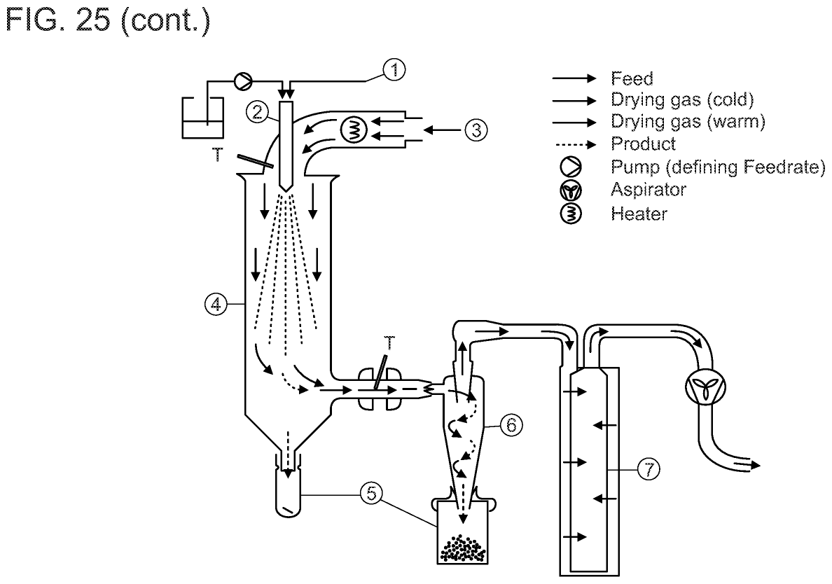

Particle size distribution of manufactured formulation #09 consistency batches are similar, and formulation #10-5 consistency batches have identical particle size distribution.
Continuous flow process allows better control of the reaction time which translates to narrower particle size distribution.
Size distribution of the particles were measured with a Sympatec HELOS laser diffraction instrument.
Generally, the average size of silica microparticles manufactured with our system, is 5±2 μm (D50, e.g., 50% of the particles are of the size indicated or smaller).
FIG. 22 shows graphs of particle size distribution from consistency batches.
FIG. 23 shows cumulative in vitro in sink dissolution of elamipretide microparticle formulations #09 (2% API load) and #10-5 (5% API load).
The continuous flow reactor system manufacturing process is scalable and was demonstrated on scale up to 10×, as shown in FIGS. 24 and 25.
The process was successfully changed from batch to continuous flow process with 2% and 5% API payload material.
pH and concentrations of the silica and API remained consistent through manufacturing process for 2% and 5% API payload material.
All manufactured microparticle formulation #09 and #10-5 batches have nearly identical PSD profiles (except for minor variation in the size of right-side shoulder for the first manufactured batch).
Batch size was successfully increased to 10×(in comparison to feasibility study batch size) and dissolution profile of the manufactured formulation remained identical to consistency batches for 2% API formulation. For the 5% API formulation, the 10×batch had a slightly lower dissolution profile.
   Temperature control for the collection vessel prevented the change in dissolution that was seen with the 5% payload material
The continuous flow process is repeatable process to produce silica-API microparticles with 2% and 5% API payload

### Example 11: Phase I Clinical Trial Data of Elamipretide in Intermediate Age-Related Macular Degeneration and High-Risk Drusen

A Phase 1 study was undertaken to assess the safety, tolerability, and feasibility of subcutaneous administration of elamipretide in patients with intermediate age-related macular degeneration (AMD) and high-risk drusen (HRD). Results of this study, entitled ReCLAIM, were published in Ophthalmology Science (M. J. Allingham, P. S. Mettu, and S. W. Cousins; Ophthalmology Science, Vol. 2, Issue 1, Mar. 2022, 100095; https://doi.org/10.1016/j.xops.2021.100095; incorporated herein by reference in its entirety).

The ReCLAIM study demonstrated that elamipretide is safe and well-tolerated in treating AMD and HRD.

### Example 12: Elamipretide Treatment Can Slow or Reverse Age-Related Visual Decline

Elamipretide has been studied for its ability to treat age-related visual impairment, as detailed in N. M. Alam, R. M. Douglas, and G. T. Prusky, Disease Models & Mechanisms (2021) 14, dmm048256. doi:10.1242/dmm.048256, which is hereby incorporated by reference in its entirety.

The authors determined that elamipretide was effective at treating—i.e. preventing as well as restoring—photopic age-related loss of spatial vision and improving the ability of the visual system to process photopic temporal information. Indeed, the study revealed behavioral evidence that elamipretide treatment rather selectively improves age-related loss of photopic visual function.

### INCORPORATION BY REFERENCE

All of the U.S. patents and U.S. and PCT published patent applications cited herein are hereby incorporated by reference.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A depot formulation comprising a biodegradable silica hydrogel composite, wherein the hydrogel composite comprises:
(i) a silica hydrogel; and
(ii) a plurality of microparticles comprising silica and an oligopeptide or a peptidomimetic compound, or a pharmaceutically acceptable salt thereof;
wherein a freebase equivalent of the oligopeptide or the peptidomimetic compound has a molecular weight less than 1,200 amu; and
the hydrogel composite is non-flowing and structurally stable when stored at rest, and shear-thinning when shear stress is applied.

2. The depot formulation of claim 1, wherein the oligopeptide or the peptidomimetic compound is positively charged in aqueous buffered solution at neutral pH (e.g., the net charge is greater than or equal to +2).

3. The depot formulation of claim 1 or 2, wherein the oligopeptide or the peptidomimetic compound is water soluble (e.g., has a water solubility of >50 mg/mL or 100 mg/mL at 25° C.).

4. The depot formulation of any one of claims 1-3, wherein the microparticles are present in the depot formulation in an amount from about 30 to about 95 wt %.

5. The depot formulation of any one of claims 1-4, wherein the microparticles are present in the depot formulation in an amount from about 45 to about 90 wt %, 50 to about 85 wt % or 75 to 90 wt %.

6. The depot formulation of any one of claims 1-5, wherein the microparticles are present in the depot formulation in an amount of about 80 wt %.

7. The depot formulation of any one of claims 1-4, wherein the microparticles are present in the depot formulation in an amount from about 30 to about 70 wt %.

8. The depot formulation of any one of claims 1-4 and 7, wherein the microparticles are present in the depot formulation in an amount from about 40 to 60 wt %.

9. The depot formulation of any one of claims 1-4 and 7-8, wherein the microparticles are present in the depot formulation in an amount of about 50 wt %.

10. The depot formulation of any one of claims 1-3, wherein the microparticles are present in the depot formulation in an amount from about 20 to about 50 wt %, or 20 to about 40 wt %.

11. The depot formulation of any one of claims 1-10, wherein the freebase equivalent of the oligopeptide or peptidomimetic is present in the depot formulation in an amount from about 0.1 wt % to about 10 wt %.

12. The depot formulation of any one of claims 1-11, wherein the freebase equivalent of the oligopeptide or peptidomimetic is present in the depot formulation in an amount from about 1 wt % to about 6 wt %.

13. The depot formulation of any one of claims 1-12, wherein the freebase equivalent of the oligopeptide or peptidomimetic is present in the depot formulation in an amount from about 1 wt % to about 3 wt %.

14. The depot formulation of any one of claims 1-12, wherein the freebase equivalent of the oligopeptide or peptidomimetic is present in the depot formulation in an amount from about 2 wt % to about 5 wt %.

15. The depot formulation of any one of claims 1-11, wherein the freebase equivalent of the oligopeptide or peptidomimetic is present in the depot formulation in an amount from about 0.25 wt % to about 2.5 wt %.

16. The depot formulation of any one of claims 1-11, wherein the freebase equivalent of the oligopeptide or peptidomimetic is present in the depot formulation in an amount of from about 0.45 wt % to about 2 wt %.

17. The depot formulation of any one of claims 1-16, wherein when shear stress is applied, the silica hydrogel composite is free flowing and can be injected from a syringe having an attached needle.

18. The depot formulation of claim 17, wherein the needle thickness is 25-30 gauge.

19. The depot formulation of any one of claims 1-18, wherein when an application of sheer stress has concluded, the silica hydrogel composite is non-flowing and structurally stable at 37° C.

20. The depot formulation of any one of claims 1-19, wherein the pH of the formulation is about 4.0 to about 7.5.

21. The depot formulation of any one of claims 1-20, wherein the pH of the formulation is about 5.0 to about 6.0.

22. The depot formulation of any one of claims 1-20, wherein the pH of the formulation is about 5.5 to about 6.5.

23. The depot formulation of any one of claims 1-22, wherein the microparticles of the depot formulation comprise more than one oligopeptide or peptidomimetic compound.

24. The depot formulation of any one of claims 1-22, wherein the microparticles comprise an oligopeptide, or a pharmaceutically acceptable salt thereof.

25. The depot formulation of claim 24, wherein the oligopeptide is selected from the group consisting of elamipretide, Compound II, Compound III, and Compound IV, and pharmaceutically acceptable salts thereof, wherein:
elamipretide has the structure: 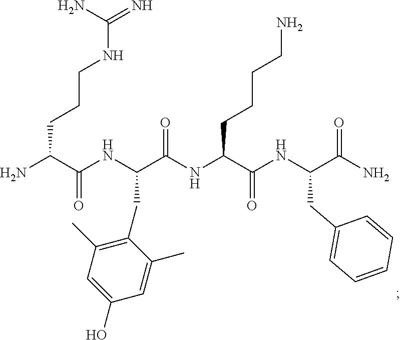 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]
Compound II has the structure: 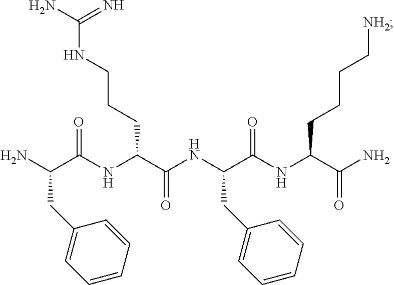 [SMILES: N=C(N)NCCC[C@@H](NC(=O)[C@@H](N)Cc1ccccc1)C(=O)N[C@@H](Cc1ccccc1)C(=O)N[C@@H](CCCCN)C(N)=O]
Compound III has the structure 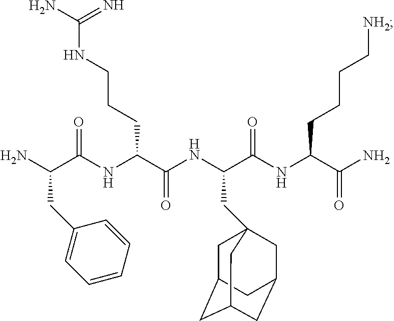 [SMILES: N=C(N)NCCC[C@@H](NC(=O)[C@@H](N)Cc1ccccc1)C(=O)N[C@@H](CC12CC3CC(CC(C3)C1)C2)C(=O)N[C@@H](CCCCN)C(N)=O]
Compound IV has the structure

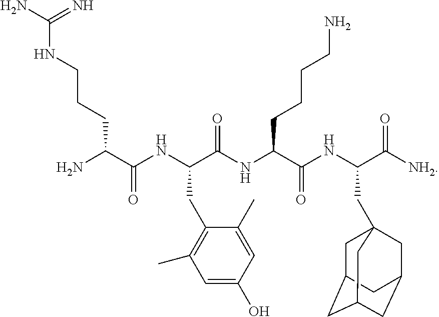 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O]

26. The depot formulation of claim 25, wherein the oligopeptide is elamipretide or a pharmaceutically acceptable salt thereof.

27. The depot formulation of claim 25, wherein the oligopeptide is Compound II or a pharmaceutically acceptable salt thereof.

28. The depot formulation of claim 25, wherein the oligopeptide is Compound III or a pharmaceutically acceptable salt thereof.

29. The depot formulation of claim 25, wherein the oligopeptide is Compound IV or a pharmaceutically acceptable salt thereof.

30. The depot formulation of any one of claims 1-22, wherein the microparticles comprise a peptidomimetic compound, or a pharmaceutically acceptable salt thereof.

31. The depot formulation of claim 30, wherein the peptidomimetic compound is: 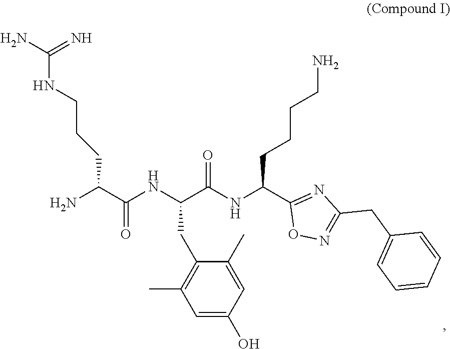 [SMILES: Cc1cc(O)cc(C)c1C[C@H](NC(=O)[C@H](N)CCCNC(=N)N)C(=O)N[C@@H](CCCCN)c1nc(Cc2ccccc2)no1]
or a pharmaceutically acceptable salt thereof.

32. The depot formulation of any one of claims 1-31, wherein the oligopeptide or peptidomimetic is mitochondria-targeted.

33. The depot formulation of any one of claims 1-32, wherein the formulation is injectable or implantable.

34. The depot formulation of any one of claims 1-32, wherein the formulation is for parenteral administration.

35. The depot formulation of claim 34, wherein the formulation is for subcutaneous, intramuscular, peritoneal, or ocular administration.

36. The depot formulation of claim 33, wherein the formulation is for intravitreal injection.

37. The depot formulation of claim 33, wherein the formulation is for topical ocular instillation.

38. The depot formulation of any one of claims 1-37, wherein the formulation is for administration from once a week to once a year, or once a month to once a year, or once a month to once every six months, or once a month to once every three months, once every three months to once every six months, once every six months to once every nine months, or once every nine months to once per year.

39. The depot formulation of any one of claims 1-37, wherein the formulation is for administration from once a month to once every six months, or once a month to once every five months, once every two months to once every five months, once every two months to once every four months, or once every two months to once every three months, once every three months to once every four months, or once about every three months.

40. The depot formulation of any one of claims 1-39, wherein the amount of the freebase equivalent of the oligopeptide or the peptidomimetic compound in the depot formulation is from 10 μg/50 μL to 100 mg/50 μL, from 10 μg/50 μL to 1000 μg/50 μL, from 10 μg/50 μL to 500 μg/50 μL, from 50 μg/50 μL to 300 μg/50 μL, or from 75 μg/50 μL to 275 μg/50 μL.

41. The depot formulation of any one of claims 1-39, wherein the amount of the freebase equivalent of the oligopeptide or the peptidomimetic compound in the depot formulation is from 250 μg/50 μL to 1000 μg/50 μL, from 150 μg/50 μL to 450 μg/50 μL, from 200 μg/50 μL to 400 μg/50 μL, from 250 μg/50 μL to 350 μg/50 μL, from 50 μg/50 μL to 250 μg/50 μL or about 270 μg/50 μL.

42. The depot formulation of any one of claims 1-41, wherein the formulation delivers a daily dose which is the freebase equivalent of the oligopeptide or the peptidomimetic compound of from 0.01 μg to 10 μg, from 0.5 μg to 5 μg, from 1 μg to 5 μg, or about 3 μg.

43. The depot formulation of any one of claims 1-42, wherein the formulation is for intravitreal injection once about every three months.

44. The depot formulation of any one of claims 1-43, wherein the formulation is a sterilized formulation.

45. The depot formulation of claim 44, wherein the sterilized formulation is a gamma ray sterilized formulation.

46. The depot formulation of any one of claims 1-45, wherein the silica hydrogel has a solid content of ≤3 wt %, ≤2 wt % or ≤1 wt %.

47. The depot formulation of any one of claims 1-45, wherein the silica particles comprise from 0.1 to 70 wt %, from 0.3 to 50 wt %, or from 1 to 15 wt % of the oligopeptide or the peptidomimetic compound.

48. The depot formulation of any one of claims 1-45, wherein the silica particles are microparticles having a diameter between 0.5 μm and 300 μm, between 0.5 μm and 100 μm, between 0.5 μm and 30 μm or between 0.5 μm and 20 μm.

49. The depot formulation of any one of claims 1-45, wherein the volume fraction of silica particles having a diameter <1 μm is <3%, <2%, or <1%.

50. The depot formulation of any one of claims 1-45, wherein the composite solid content is from 10 wt % to 75 wt %, from 15 wt % to 60 wt % or from 25 wt % to 55 wt %.

51. The depot formulation of any of claims 1-45, wherein the complex modulus measured under small angle oscillatory shear in the linear viscoelastic region is <2400 kPa, <1200 kPa or <600 kPa.

52. The depot formulation of any of claims 1-45, wherein the loss factor (i.e., viscous modulus/elastic modulus) is <1, <0.8, or <0.6.

53. The depot formulation of any of claims 1-45, wherein the viscosity is 10-50 Pas measured with a shear rate of 10-50 s⁻¹, the viscosity is 0.4-1.5 Pas measured with a shear rate of 200-210 s⁻¹, and 0.1-0.4 Pas measured with a shear rate of 600-610 s⁻¹.

54. The depot formulation of any of claims 1-53, wherein the formulation is prepackaged in a syringe and needle configuration for single-use intravitreal injection.

55. The depot formulation of any of claims 1-54, wherein the plurality of microparticles comprising silica and an oligopeptide or a peptidomimetic compound, or a pharmaceutically acceptable salt thereof, is formed using a continuous flow process.

56. The depot formulation of claim 55, wherein the continuous flow process further comprises use of a spray dryer to generate the plurality of microparticles comprising silica and an oligopeptide or a peptidomimetic compound, or a pharmaceutically acceptable salt thereof.

57. The depot formulation of claim 55 or 56, wherein the continuous flow process further comprises use of an in-line mixer.

58. A method of treating, inhibiting, ameliorating or delaying the onset of a disease, disorder, or condition comprising administering to a subject in need thereof an effective amount of the depot formulation of any one of claims 1-57.

59. The method of claim 58, wherein the disease, disorder, or condition is Leber's Hereditary Optic Neuropathy.

60. The method of claim 58, wherein the disease, disorder or condition is Fuchs endothelial corneal dystrophy (FECD), age-related macular degeneration (AMD), glaucoma, optic neuropathy, retinopathy, diabetic macular edema, diabetic retinopathy, retinal telangiectasia, or retinitis pigmentosa.

61. The method of claim 58, wherein the disease, disorder or condition is AMD, including neovascular AMD (wet AMD) and retinal geographic atrophy (dry AMD).

62. A prefilled syringe comprising the depot formulation of any one of claims 1-57, said prefilled syringe optionally being sterilized by exposure to gamma radiation.

63. A method of making a depot formulation of any one of claims 1-57, comprising combining:
a) silica particles comprising an oligopeptide or a peptidomimetic compound, or a pharmaceutically acceptable salt thereof, and having a maximum diameter of ≤1000 μm, optionally as a suspension wherein a freebase equivalent of the oligopeptide or the peptidomimetic compound has a molecular weight less than 1,200 amu; and
b) a silica hydrogel;
wherein:
i) the silica hydrogel has a solid content of ≤50 wt %;
ii) the silica particles comprise polymerized alkoxysilanes;
iii) the depot formulation comprises up to 85 wt % of said silica particles; and
iv) said hydrogel composite is non-flowing and structurally stable when stored at rest and shear-thinning when shear stress is applied by injection.

64. The method of claim 63, further comprising combining a silica sol with a solution comprising the oligopeptide or the peptidomimetic compound, or a pharmaceutically acceptable salt thereof, thereby producing a mixture comprising the silica sol and the oligopeptide or a peptidomimetic compound, or a pharmaceutically acceptable salt thereof.

65. The method of claim 64, further comprising combining tetraethyl orthosilicate (TEOS), water and hydrochloric acid, and optionally an organic co-solvent (e.g. ethanol), thereby producing the silica sol.

66. The method of claim 64 or 65, further comprising combining the oligopeptide or the peptidomimetic compound, or a pharmaceutically acceptable salt thereof, with water and optionally an organic co-solvent (e.g. ethanol), thereby producing the solution comprising the oligopeptide or the peptidomimetic compound, or a pharmaceutically acceptable salt thereof.

67. The method of any of claims 64-66, wherein combining the silica sol and the solution comprising the oligopeptide or the peptidomimetic compound, or a pharmaceutically acceptable salt thereof, is performed in a continuous flow process.

68. The method of claim 67, wherein the step of combining the silica sol and the solution comprising the oligopeptide or the peptidomimetic compound, or a pharmaceutically acceptable salt thereof, further comprises use of an in-line mixer.

69. The method of any one of claims 64-68, wherein the step of combining the silica sol and the solution comprising the oligopeptide or the peptidomimetic compound, or a pharmaceutically acceptable salt thereof, further comprises use of a pump.

70. The method of any one of claims 69-69, further comprising spray drying the mixture of the silica sol and the solution comprising the oligopeptide or the peptidomimetic compound, or a pharmaceutically acceptable salt thereof, thereby forming the silica particles.

71. The method of any one of claims 63-70, wherein formulation is packaged into a syringe, optionally with an attached needle.

72. The method of any one of claims 63-71, further comprising sterilizing the depot formulation.

73. The method of claim 72, wherein the step of sterilizing the depot formulation occurs by radiation sterilization.

74. The method of claim 73, wherein the radiation sterilization comprises gamma-ray radiation sterilization.

75. The method of any one of claims 63-74, wherein the oligopeptide is elamipretide: 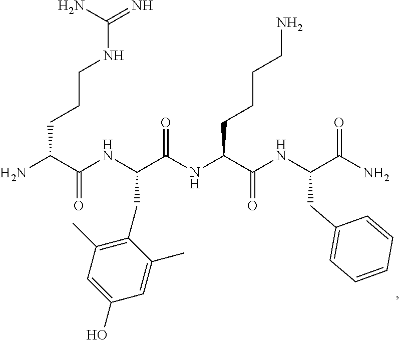 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]
or a pharmaceutically acceptable salt thereof.

76. The method of any one of claims 63-74, wherein the oligopeptide is Compound II: 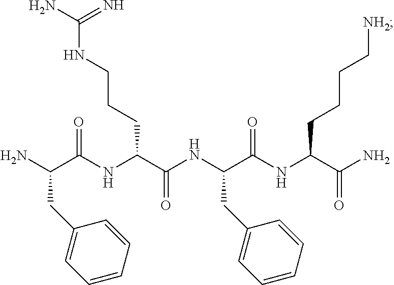 [SMILES: N=C(N)NCCC[C@@H](NC(=O)[C@@H](N)Cc1ccccc1)C(=O)N[C@@H](Cc1ccccc1)C(=O)N[C@@H](CCCCN)C(N)=O]
or a pharmaceutically acceptable salt thereof.

77. The method of any one of claims 63-74, wherein the oligopeptide is Compound III: 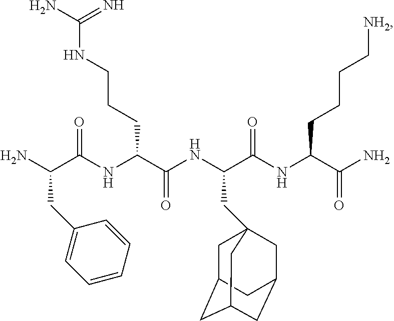 [SMILES: N=C(N)NCCC[C@@H](NC(=O)[C@@H](N)Cc1ccccc1)C(=O)N[C@@H](CC12CC3CC(CC(C3)C1)C2)C(=O)N[C@@H](CCCCN)C(N)=O]
or a pharmaceutically acceptable salt thereof.

78. The method of any one of claims 63-74, wherein the oligopeptide is Compound IV: 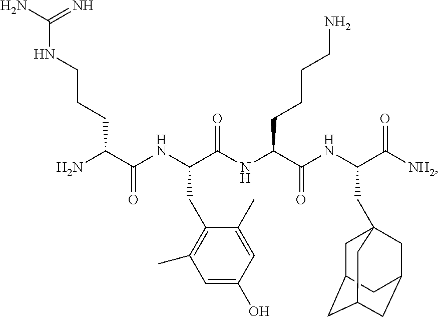 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O]
or a pharmaceutically acceptable salt thereof.

79. The method of any one of claims 63-74, wherein the peptidomimetic is Compound I: 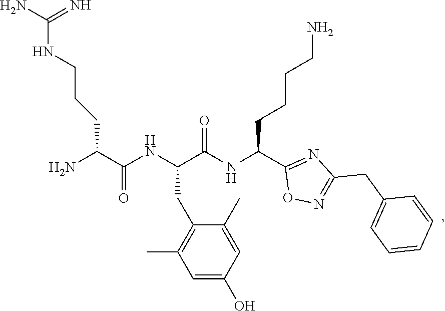 [SMILES: Cc1cc(O)cc(C)c1C[C@H](NC(=O)[C@H](N)CCCNC(=N)N)C(=O)N[C@@H](CCCCN)c1nc(Cc2ccccc2)no1]
or a pharmaceutically acceptable salt thereof.
